(19) United States
(12) Patent Application Publication
(10) Pub. No.: US 2006/0041958 A1
(43) Pub. Date: Feb. 23, 2006

(54) Plant gene

(76) Inventors: Melinka Butenko, Oslo (NO); Reidunn Aalen, Oslo (NO)

Correspondence Address: FROMMER LAWRENCE & HAUG, 745 FIFTH AVENUE- 10TH FL., NEW YORK, NY, 10151, US

(21) Appl. No.: 11/159,667

(22) Filed: Jun. 23, 2005

Related U.S. Application Data
(63) Continuation-in-part of application No. PCT/NO03/00428, filed on Dec. 17, 2003.

(30) Foreign Application Priority Data
Dec. 23, 2002 (GB) 0230039.0
Jun. 13, 2003 (GB) 0313773.4

(51) Int. Cl. A01H 1/00; C12N 15/82

(52) U.S. Cl. 800/287; 435/468

(57) ABSTRACT

A process is described for decreasing organ abscission in plants by modifying the IDA gene, a homologue, fragment, or derivative thereof, or the expression thereof.

### REFERENCE TO RELATED APPLICATIONS

This application is a continuation-in-part of International Patent Application PCT/NO2003/000428 filed Dec. 17, 2003 and published as WO 04/057004 on Jul. 8, 2004, which claims priority from Great Britain patent application numbers 0230039.0 filed Dec. 23, 2002 and 0313773.4 filed Jun. 13, 2003. Each of the above referenced applications, and each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

The present invention relates to new plant genes and their use in controlling abscission of organs in plants.

The present invention also relates to new plants having delayed abscission.

In particular the present invention relates to producing plants with longer lasting flowers or other ornamental or decorative organs including leaves.

Preferred plants according to the invention include flowering plants such as lotus, tulips, roses, poinsettias or trees eg. Christmas trees.

Abscission, a physiologically determined program of cell separation, aids in the removal of senescent or damaged organs, and in the shedding of organs which are unwanted or no longer have a function for the plant (1). The process requires the formation of an abscission zone (AZ) at the base of the organ to be shed (2). In the AZ, enzymatic hydrolysis leads to the dissolution of the cell walls between adjacent living cells resulting in organ detachment (3). As the purpose of the flower is to facilitate pollination, it is usually abscised following fertilization.

Ethylene has long been associated with regulation of abscission, and has been shown to accelerate the abscission process in many plants (4). Recent reports on delayed floral organ abscission, e.g. one involving the protein level of the leucine-rich repeat (LRR) receptor-like kinase (RLK) HAESA and another the over-expression of the MADS domain factor AGL15 (5,6), question ethylene as the sole inducer and regulator of the gene expression program that causes separation. The ethylene-insensitive Arabidopsis thaliana mutants etr1 (7) and ein2 (8) show a considerable delay in floral abscission, but comparative studies demonstrate that these mutants virtually go through the same developmental progression as wild type (wt) plants (1,9).

WO 02/061042 describes a gene (NEVERSHED) on chromosome 5 of Arabidopsis and its use in a method of preventing floral abscission by mutating the ARF GAP domain of the NEVERSHED gene.

In one broad aspect the present invention relates to a method of decreasing organ abscission in a plant, comprising reducing the expression level of a gene in a plant.

The term “reducing the expression level of a gene” includes one or more of:
reducing or altering or eliminating expression of the coding region of the gene;
reducing or altering or eliminating the activity of the protein encoded by the coding region of the gene;
reducing or altering or eliminating the affect of the gene regulatory sequences—i.e. so that they have a reduced activity or no activity at all;
reducing or altering or eliminating the affect of the gene promoter sequence—i.e. so that it has a reduced activity or no activity at all.

In one preferred aspect, the present invention relates to a method of decreasing organ abscission in a plant, comprising reducing the expression level of a gene in a plant, wherein the gene is the IDA gene or a mimetic thereof.

In another broad aspect the present invention relates to plants that can exhibit decreased organ abscission.

In another broad aspect the present invention relates to one or more modified gene(s) that can cause a plant to exhibit decreased organ abscission.

The term “modified gene(s)” includes one or more of:
a gene having a reduced or altered or eliminated expression of the coding region of the gene;
a gene having a reduced or altered or eliminated activity of the protein encoded by the coding region of the gene;
a gene having a reduced or altered or eliminated affect of the gene regulatory sequences—i.e. so that they have a reduced activity or no activity at all;
a gene having a reduced or altered or eliminated affect of the gene promoter sequence—i.e. so that it has a reduced activity or no activity at all.

Here, the modified genes may be mutated or silenced gene(s) that can cause a plant to exhibit decreased organ abscission.

The term “gene” as used herein means a nucleotide sequence comprising one or more regulatory sequence(s) and/or one or more coding sequence(s) and/or one or more non-coding region(s).

In a highly preferred aspect, the term “gene” means a nucleotide sequence comprising at least one or more regulatory sequence(s).

In a highly preferred aspect, the term “gene” means a nucleotide sequence comprising at least a promoter sequence.

Preferably, the present invention relates to a mutated gene. The mutation may be one or more of: one or more of substitution(s), one or more of deletion(s), one or more of insertion(s) of sequences.

Preferably, the mutation is at least one substitution and/or at least one deletion.

The mutation may be in the regulatory region(s) and/or in the coding region(s).

Preferably the mutation in the gene comprises at least a mutation in a portion of a regulatory region.

The term “regulatory sequences” includes promoters and enhancers and other expression regulation signals.

The mutations may be in adjacent regions and/or in remote regions.

The mutation(s) cause lower—or even eliminate—expression of the gene. In this respect, the mutation may be in the promoter region so as to prevent the promoter acting as a promoter. In the alternative and/or in addition, the mutation may be in the coding region so that expression of the coding region is reduced (or even eliminated) and/or expression leads to a non-functional protein. Here, the term “non-functional” means a protein that does not have the same type and/or level of activity as the protein encoded by the non-mutated coding sequence. Preferably, the term “non-functional” means a protein that does not have any activity.

In a preferred aspect, the mutation in the gene comprises at least a mutation in a portion of the regulatory sequence so as to cause very low expression—preferably no expression—of the coding sequence.

In a highly preferred aspect, the mutation in the gene comprises at least a mutation in a portion of the promoter sequence.

The term “promoter” is used in the normal sense of the art, e.g. an RNA polymerase binding site.

In a highly preferred aspect, the mutation in the gene comprises at least a mutation in a portion of the promoter sequence so as to inactivate the promoter. The term “inactivate” means at least reduce the activity of the promoter, preferably at least substantially inactivate the activity. In a more preferred embodiment, the term “inactivate” means complete inactivation.

In another broad aspect the present invention relates to vectors carrying nucleotide sequences that can cause a plant to exhibit decreased organ abscission.

In another broad aspect the present invention relates to a mutated regulatory sequence that can cause a plant to exhibit decreased organ abscission.

In another broad aspect the present invention relates to a process for decreasing organ abscission in plants by modifying the IDA gene, a homologue, fragment, or derivative thereof, or the expression thereof.

The present invention will now be described with respect to general and specific embodiments.

In the following commentary, reference is made to the accompanying sequences.

SEQ ID NO. 1: Which is the sequence of the IDA gene. This DNA sequence was used to complement the ida mutation. The start and stop codon of the IDA gene is indicated by bold letters (and in larger font). The first and last base pair of the IDA mRNA sequence are underlined (and in larger font).

SEQ ID NO. 2: Which is the IDA cDNA sequence (accession number AY087883). The coding sequence in the cDNA is base pairs 98-331, see bold start codon (atg) (and in larger font) and bold stop codon (and in larger font).

SEQ ID NO. 3: Which is the IDA protein, amino acid sequence (accession number AM65435)

SEQ ID NO. 4: Which is AtIDL2

SEQ ID NO. 5: Which is AtIDL3.

SEQ ID NO. 6: Which is AtIDL4.

SEQ ID NO. 7: Which is AtIDL5:

SEQ ID NO. 8: Which presents the upstream portion of the IDA gene. This sequence comprises the promoter region. In a highly preferred aspect of the present invention, the mutation is made or is present in the promoter portion of this sequence.

SEQ ID NO. 9: which presents the upstream portion of the IDA gene. This sequence comprises the promoter region. The primers used to amplify the promoter (used to demonstrate expression in the abscission zone) are underlined and are in bold. In a highly preferred aspect of the present invention, the mutation is made or is present in the promoter portion of this sequence.

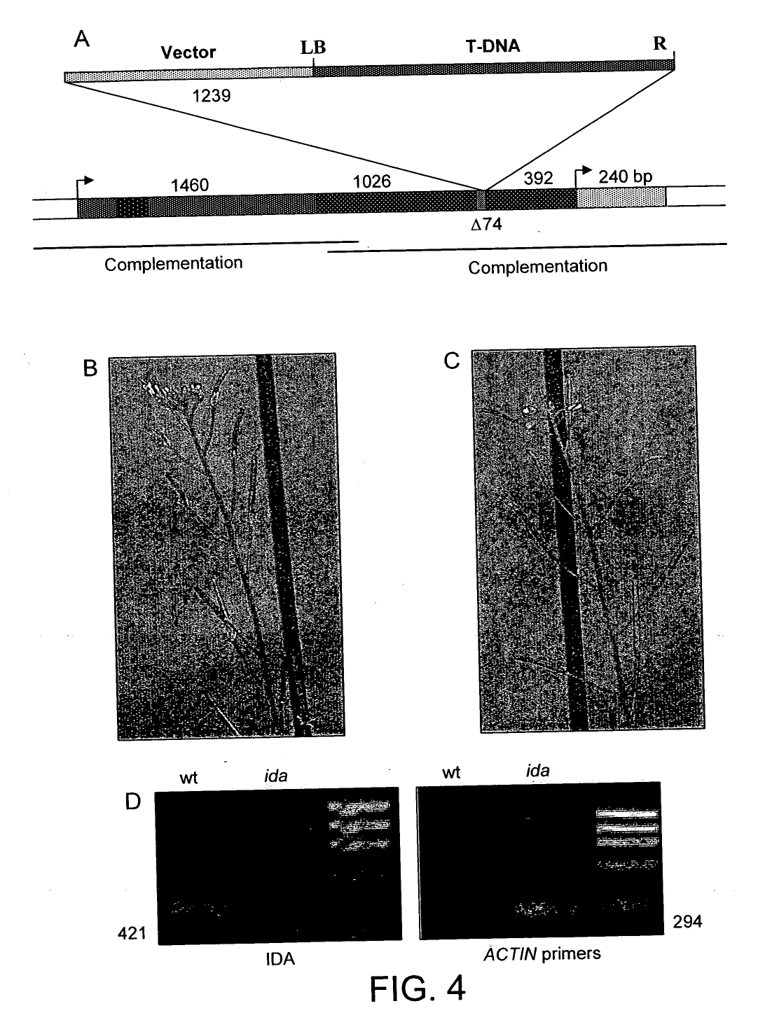

SEQ ID NO. 10: which presents the upstream portion of the IDA gene. This sequence comprises the promoter region. In a highly preferred aspect, the fragment of the promoter that is deleted in the ida mutant due to the T-DNA insertion (FIG. 4) is given in italics and in bold.

SEQ ID NO. 11: which presents the IDA gene. The position of the T-DNA insertion in the SALK line 133209 (discussed below) is between the a and t nucleotides in the coding region given in italics and bold and by underlining.

SEQ ID NO. 12: Which is the coding sequence for IDA—including the start and stop codons.

Specific aspects of the present invention include:

A method of decreasing organ abscission in a plant, comprising reducing the expression level of a gene in a plant, wherein the gene is the IDA gene or a mimetic thereof.

A modified nucleotide sequence; wherein when a plant comprises or is transformed with said modified nucleotide sequence said plant exhibits decreased organ abscission.

A nucleotide sequence; wherein the nucleotide sequence is all or part of a sequence selected from: AY087883, AtIDL1, gene At3g25655, tomato (LeIDL1, ac. no AI779570, lotus (LjIDL1, ac. NO. AW719486), soybean (GmIDL1, ac.NO. Bq630646), black locust (RpIDL1, ac.NO. BI642538), maize (ZmIDL1, ac.NO. BI430572), poplar (PTIDL1, ac.NO. BU889756), and wheat (TaIDL1, ac.no BM135459), a nucleotide sequence that includes a coing sequence for a C terminal motif PpSa/gPSk/rk/rHN, a nucleotide sequence that includes a coing sequence for an N terminal hydrophobic signal peptide, a nucleotide sequence that includes a coing sequence for a C terminal motif v/iPpSa/gPSK/rk/rHN and a coding sequence for an N terminal hydrophobic signal peptide; wherein said nucleotide sequence is a mutation of any of said sequence; and wherein when a plant comprises or is transformed with said nucleotide sequence said plant exhibits decreased organ abscission.

A nucleotide sequence that is a mutation of the sequence shown as any one of SEQ ID No. 1, SEQ ID No. 2, SEQ ID No. 8, SEQ ID No. 9, SEQ ID No. 10 or SEQ ID No. 11 or a mimetic of any thereof; wherein when a plant comprises or is transformed with said nucleotide sequence said plant exhibits decreased organ abscission.

A nucleotide sequence that is a mutation of the sequence shown as any one of SEQ ID No. 1, SEQ ID No. 2, SEQ ID No. 8, SEQ ID No. 9, SEQ ID No. 10 or SEQ ID No. 11; wherein when a plant comprises or is transformed with said nucleotide sequence said plant exhibits decreased organ abscission.

A construct comprising the invention according to the aspects presented herein.

A vector comprising the invention according to the aspects presented herein.

An expression vector comprising the invention according to the aspects presented herein.

A transformation vector comprising the invention according to the aspects presented herein.

A host cell comprising the invention according to the aspects presented herein.

An organ comprising the invention according to the aspects presented herein.

An organism comprising the invention according to the aspects presented herein.

A transformed plant comprising the invention according to the aspects presented herein.

A method comprising transforming a plant with the invention according to the aspects presented herein.

Use of the invention according to the aspects presented herein in the preparation of a transformed plant to decrease organ abscission.

A process for decreasing organ abscission in plants by modifying the IDA gene, a homologue, fragment, or derivative thereof, or the expression thereof.

A nucleotide sequence, modified nucleotide sequence; wherein when a plant comprises or is transformed with said nucleotide sequence said plant exhibits decreased organ abscission.

Use of a promoter sequence comprising all or part of the nucleotide sequence presented as SEQ ID No. 1, SEQ ID No. 2, SEQ ID No. 8, SEQ ID No. 9, SEQ ID No. 10 or SEQ ID No. 11 to express an heterolgous coding sequence.

Use according to claim 31 wherein said heterologous coding sequence is a mutated coding sequence corresponding to the coding sequence naturally associated with said promoter sequence.

An isolated and/or purified nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified construct comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified vector comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified expression vector comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified transformation vector comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified transformed cell comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11; wherein said cell is not a naturally occuring cell.

An isolated and/or purified nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified construct comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified vector comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified expression vector comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified transformation vector comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

An isolated and/or purified transformed cell comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11; wherein said cell is not a naturally occuring cell.

The term “mimetic” includes variants of the reference sequence (e.g the IDA gene) as well as other sequences that have a similar function; wherein those other sequences may have a sequence identity that is close to that of the reference gene (e.g. the IDA gene—see SEQ ID No. 1). With respect to the IDA gene and mimetics thereof, the mimetic sequences may be referred to herein as being “IDA like” or “IDL”.

As mentioned above, the mutation in the gene comprises at least a mutation in a portion of the regulatory sequence so as to cause very low expression—preferably no expression—of the coding sequence. In a highly preferred aspect, the mutation in the gene comprises at least a mutation in a portion of the promoter sequence.

In a highly preferred aspect, the mutation in the gene comprises at least a mutation in a portion of the promoter sequence of the nucleotide sequence as shown in SEQ ID No. 1 or a variant, homologue, fragment or derivative thereof. Here, the mutated nucleotide sequence causes (such as by the expression thereof) a plant to exhibit decreased organ abscission.

In a highly preferred aspect, the mutation in the gene comprises at least a mutation in a portion of the promoter sequence as shown in SEQ ID NO. 1, 8 or 9—or a mimetic thereof. The term mimetic means a sequence that is similar in sequence homology or identity and which can have the same affect as the mutated sequence—i.e. can lead to decreased organ abscission in a plant transformed with same.

In a specific embodiment as herein described, a mutation is introduced via T-DNA insertion in a promoter region of a gene. However, as will become apparent, other techniques for creating mutations will be readily apparent to those skilled in the art. It is also to be understood that the mutated genes can be prepared de novo using, for example, recombinant DNA techniques as opposed to using T-DNA insertion techniques.

In a specific embodiment as herein described, a mutation is introduced into gene Atg68765. However, as will become apparent, other genes can be used.

In a highly specific embodiment as herein described, the mutation is situated upstream of the atg start codon and generates a deletion. However, as will become apparent, other mutations can be used.

In a highly specific embodiment as herein described, a mutation is introduced via a T-DNA insertion in the promoter of the gene Atg68765. This is shown schematically in FIG. 4, where it is shown that the insertion i situated 392 bp upstream of the atg start codon and has generated a 74 bp deletion. In addition, ID sequence 1 indicates in italics the deletion, e.g. the position of the insertion. The insertion consists of the T-DNA of the plasmid pMHA2 and 1239 bp vector backbone sequence of this plasmid. However, as will become apparent, other mutations and/or other genes can be used.

In an alternative and/or additional aspect, the mutation in the gene comprises at least a mutation in a portion of the coding region wherein expression of the coding region is reduced—or eliminated—mutated nucleotide sequence causes (such as by the expression thereof) a plant to exhibit decreased organ abscission.

In an alternative and/or an additional aspect, expression of the coding sequence is reduced or eliminated through use of interfering moieties—such as anti-sense DNA. Another example is RNA interference techniques. Here, the interfering moieties cause a plant to exhibit decreased organ abscission.

Preferably said gene includes a coding sequence for a C terminal motif PpSa/gPSk/rk/rHN.

Preferably said gene includes a coding sequence for a N terminal hydrophobic signal peptide.

Preferably said gene includes a coding sequence for a C terminal motif v/iPpSa/gPSK/rk/rHN and a coding sequence for an N terminal hydrophobic signal peptide.

Preferably said gene is selected from: AY087883, AtIDL1, gene At3g25655, tomato (LeIDL1, ac. no AI779570, lotus (LJIDL1, ac. NO. AW719486), soybean (GmIDL1, ac.NO. Bq630646), black locust (RpIDL1, ac.NO. BI642538), maize (ZmIDL1, ac.NO. BI430572), poplar (PtID1, ac.NO. BU889756), and wheat (TaIDL1, ac.no BM135459)

Preferably said gene is or comprises all or part of the nucleotide sequence presented as any one of SEQ ID No. 1, SEQ ID No. 2, SEQ ID No. 8, SEQ ID No. 9, SEQ ID No. 10 or SEQ ID No. 11 or a variant of any thereof or a homologue of any thereof.

Preferably said gene is or comprises all or part of the nucleotide sequence presented as any one of SEQ ID No. 1, SEQ ID No. 2, SEQ ID No. 8, SEQ ID No. 9, SEQ ID No. 10 or SEQ ID No. 11.

The expression level may be reduced by any suitable means—such as by insertions of disruptive sequences and/or deletions of important sequence regions and/or modification of regions and/or use of moieties that affect the expression levels of a gene.

Preferably said expression level is reduced by mutating said gene.

Preferably said expression level is reduced by mutating a regulatory region of said said gene.

Preferably said expression level is reduced by mutating the promoter region of said said gene.

Preferably said expression level is reduced by mutating the promoter region of said said gene by use of T-DNA insertion techniques.

Preferably said sequence is a mutation of the sequence presented as any one of SEQ ID No. 1, SEQ ID No. 2, SEQ ID No. 8, SEQ ID No. 9, SEQ ID No. 10 or SEQ ID No. 11 or a variant of any thereof or a homologue of any thereof or a mimetic of any thereof.

Preferably said sequence is a mutation of the sequence shown as any one of SEQ ID No. 1, SEQ ID No. 2, SEQ ID No. 8, SEQ ID No. 9, SEQ ID No. 10 or SEQ ID No. 11.

The technical aspects of the present invention are not limited to a specific plant type—as will become apparent herein.

Preferably said plant is a flowering plant or tree.

The promoter sequence may be operably linked to either the naturally associated coding region or a coding region that is not naturally associated with said promoter.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

As used with reference to the present invention, the terms “produce”, “producing”, “produced”, “producable” are synonymous with the respective terms “prepare”, “preparing”, “prepared”, “generated” and “preparable”.

As used with reference to the present invention, the terms “expression”, “expresses”, “expressed” and “expressable” are synonymous with the respective terms “tanscription”, “transcribes”, “transcribed” and “transcribable”.

As used with reference to the present invention, the terms “transformation” and “transfection” refer to a method of introducing nucleic acid sequences into hosts, host cells, tissues or organs.

Other aspects concerning the nucleotide sequence which can be used in the present invention include: a construct comprising the sequences of the present invention; a vector comprising the sequences for use in the present invention; a plasmid comprising the sequences for use in the present invention; a transformed cell comprising the sequences for use in the present invention; a transformed tissue comprising the sequences for use in the present invention; a transformed organ comprising the sequences for use in the present invention; a transformed host comprising the sequences for use in the present invention; a transformed organism comprising the sequences for use in the present invention. The present invention also encompasses methods of expressing the nucleotide sequence for use in the present invention using the same, such as expression in a host cell; including methods for transferring same. The present invention further encompasses methods of isolating the nucleotide sequence, such as isolating from a host cell.

The present inventors have investigated the control of floral organ abscission by a new Arabidopsis gene INFLORESCENCE DEFICIENT IN ABSCISSION (IDA). The present inventors have identified an Arabidopsis mutant, inflorescence deficient in abscission (ida), in which floral organs remain attached to the plant body after the shedding of mature seeds, even though a floral abscission zone develops. In contrast to etr1 and ein2, the ida mutant is sensitive to ethylene. The IDA gene, complementing the mutation, encodes a small protein with a N-terminal export signal, suggesting the IDA protein to be a receptor ligand—wherein the receptor may be involved in the developmental control of floral abscission. IDA genes have been found by the present inventors in a number of commercially relevant plant species (these are referred to IDA like or IDL in the examples herein). The present inventors have found that genetic modification of the IDA gene of a plant can be modified so that the plant exhibits decreased organ abscission.

Accordingly, in one aspect the present invention provides a modified plant comprising a nucleotide sequence as shown in ID Seq. 1 or a variant, homologue, fragment or derivative thereof, wherein said sequence or the expression thereof has been modified so that the plant exhibits decreased organ abscission.

Preferred sequences for modification are selected from (AtIDL1, gene At3g25655), tomato (LeIDL1, ac. no AI779570), lotus (LjIDL1, ac. NO. AW719486), soybean (GmIDL1, ac. NO. Bq630646), black locust (RpIDL1, ac.NO. BI642538), maize (ZmIDL1, ac.NO. BI430572), poplar (PtIDL1, ac.NO. BU889756), and wheat (TaIDL1, ac.no BM135459).

Preferably the nucleotide sequence includes a coding sequence for a C terminal motif PpSa/gPSk/rk/rHN, or a coding sequence for a N terminal hydrophobic signal peptide or more preferably a coding sequence for a C terminal motif v/iPpSa/gPSK/rk/rHN and a coding sequence for an N terminal hydrophobic signal peptide.

A preferred sequence is SEQ ID No. 2 (AY087883).

In a preferred aspect of the present invention, the decreased organ abscission is or relates to a flower or parts thereof. Preferably the plant is a flowering plant or tree, for example Arabidopsis thaliana.

Particularly preferred plants include flowers such as crocus (crocus spp.), tulip (eg. Haemanthus spp.), cyclamen (cyclamen spp.), poinsettia (Euphorbia Pulcherrima), lotus (e.g. Nelumbo) and rose (Rosa spp.), and trees such as poplar (populus) and Christmas tree (e.g. Blandfordia grandiflora, Nuytsiafloribunda, Pica abies).

In a further aspect the present invention provides a seed or other propagating material, or a flower from a plant according to the present invention.

In a preferred aspect the present invention provides a process of preventing organ loss in a plant comprising modifying the sequence or expression of a sequence defined above. Suitably the process of modification is by mutation or deletion. The modification can be of a promoter or other regulatory sequence. Suitably the modification is achieved by the use of an antisense construct or a RNA interference construct.

The present invention also provides the use of recombinant or isolated nucleotide sequence according to the invention as claimed in the control of plant abscission.

In a further aspect the present invention also provides an isolated nucleotide sequence comprising the sequence as shown in SEQ ID No. 1 or a variant, homologue, fragment or derivative thereof.

Preferably the sequence is selected from SEQ ID No. 2 [AY087883] and the sequences listed in Table 1 including AtIDL1, gene At3g25655, tomato (LeIDL1, ac. no AI779570), lotus (LjIDL1, ac. NO. AW719486), soybean (GMIDL1, ac.NO. Bq630646), black locust (RpIDL1, ac.NO. BI642538), maize (ZmIDL1, ac.NO. BI430572), poplar (PtIDL1, ac.NO. BU889756), and wheat (TaIDL1, ac.no BM135459). Particularly preferred is the sequence comprising ID Seq 2 [AY087883].

In a further aspect the present invention provides a nucleotide sequence which is antisense to the nucleotide sequence of the invention. Antisense technology has been used previously, for example in controlling the ripening of fruits such as the tomato.

In a preferred aspect the present invention provides the use of an isolated nucleotide sequence as defined herein in the control of plant organ abscission.

The term ‘isolated’ means that the sequence is at least substantially free from at least one other component with which the sequence is normally associated in nature and as found in nature.

In one aspect, preferably the sequence is in a purified form. The term “purified” means that the sequence is in a relatively pure state—e.g. at least about 90% pure, or at least about 95% pure or at least about 98% pure.

The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” in relation to the present invention includes genomic DNA, cDNA, synthetic DNA, and RNA. Preferably it means DNA.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”.

Typically, the nucleotide sequence encompassed by scope of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers MH et al., (1980) Nuc Acids Res Symp Ser 215-23 and Horn T et al., (1980) Nuc Acids Res Symp Ser 225-232).

A nucleotide sequence which has the specific properties as defined herein or a sequence which is suitable for modification may be identified and/or isolated and/or purified from any cell or organism. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from a suitable organism. A labelled oligonucleotide probe containing sequences homologous to another known gene could be used to identify suitable clones. In the latter case, hybridisation and washing conditions of lower stringency are used.

Alternatively, suitable clones could be identified by inserting fragments of genomic DNA into an expression vector, such as a plasmid, transforming enzyme-negative bacteria with the resulting genomic DNA library, and then plating the transformed bacteria onto agar plates containing a substrate allowing clones to be identified.

In a yet further alternative, the nucleotide sequence may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487491).

Due to degeneracy in the genetic code, nucleotide sequences may be readily produced in which the triplet codon usage, for some or all of the amino acids encoded by the original nucleotide sequence, has been changed thereby producing a nucleotide sequence with low homology to the original nucleotide sequence but which encodes the same, or a variant, amino acid sequence as encoded by the original nucleotide sequence. For example, for most amino acids the degeneracy of the genetic code is at the third position in the triplet codon (wobble position) (for reference see Stryer, Lubert, Biochemistry, Third Edition, Freeman Press, ISBN 0-7167-1920-7) therefore, a nucleotide sequence in which all triplet codons have been “wobbled” in the third position would be about 66% identical to the original nucleotide sequence however, the amended nucleotide sequence would encode for the same, or a variant, primary amino acid sequence as the original nucleotide sequence. Therefore, the present invention further relates to any nucleotide sequence that has alternative triplet codon usage for at least one amino acid encoding triplet codon, but which encodes the same, or a variant, polypeptide sequence as the polypeptide sequence encoded by the original nucleotide sequence.

Furthermore, specific organisms typically have a bias as to which triplet codons are used to encode amino acids. Preferred codon usage tables are widely available, and can be used to prepare codon optimised genes. Such codon optimisation techniques are routinely used to optimise expression of transgenes in a heterologous host.

An homologous sequence can be taken to include a nucleotide sequence which may be at least 75, 80, 85 or 90% identical, preferably at least 95, 96, 97, 98 or 99% identical to a nucleotide sequence of the present invention.

The term “nucleotide sequence” in relation to the present invention includes genomic DNA, cDNA, synthetic DNA, and RNA. Preferably it means DNA, more preferably cDNA sequence coding for the present invention.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of sources. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector.

Also useful in the invention are nucleotides sequences or fragments thereof which hybridise under stringent conditions with nucleotides defined above. For example fragments are useful for probing a gene library of a plant of interest for similar genes involved in abscission. Typical probes are 11-13 nucleotides in length. Examples of such sequences are nucleotide sequences that can hybridise to all or part of the nucleotide sequence presented as SEQ ID No. 1, SEQ ID No. 2, SEQ ID No. 8, SEQ ID No. 9, SEQ ID No. 10 or SEQ ID No. 11. Further examples of such sequences are nucleotide sequences that are complementary to all or part of the nucleotide sequence presented as SEQ ID No. 1, SEQ ID No. 2, SEQ ID No. 8, SEQ ID No. 9, SEQ ID No. 10 or SEQ ID No. 11.

In a preferred aspect, the present invention provides a sequence which is a promoter or other regulatory sequence.

Preferably the nucleotide sequence can be in the form of a vector. Vectors may be, for example, a plasmid or cosmid. The vector may be a phage. A prefered plasmid is a Ti plasmid. Preferably vectors include selectable markers so that transformed or transfixed cells can be identified.

In a preferred aspect the present invention provides a host cell transfected or transformed with a nucleotide sequence as described herein.

In a further aspect the present invention provides a plant cell including a nucleotide sequence as claimed herein.

Suitable methods for transforming plant cells is by use of any ways known to the skilled person in the area of plant molecular biology. For example sequences can be introduced into plant cells using Ti plasmids of Agrobacterium tumefaciens, using electroporation, microinjection, microprojection (biolistics), liposomes. The selection of the vector and the method of transformation will depend on the plant species to be transformed.

For example, methods for transforming tulips can be found in: Plant Cell Tissue and Organ Culture (1999) 58 (3) 213-217, Chauvin et al. (Effects of gelding agents on in vitro regeneration and kanamycin efficiency as a selective agent in plant transformation procedures, and Plant Cell reports (1992) 11 (2) 76-80, Wilmink et al., Expression of the Gus-gene in the monocot tulip after introduction by particle bombardment and Agrobacterium.

The present invention also provides a plant, or a part thereof, comprising cells as claimed herein.

In a further aspect the present invention provides an isolated amino acid sequence comprising the sequence as shown in SEQ ID No. 3 or a sequence substantially homologous thereto, or a fragment thereof.

Here, the term “homologue” means an entity having a certain homology. Here, the term “homology” can be equated with “identity”. Suitably, a homologous sequence is taken to include an amino acid sequence which may be at least 75, 80, 85 or 90% identical, preferably at least 95, 96, 97, 98 or 99% identical to the subject sequence.

Typically, the homologues will comprise the same active sites etc. as the subject sequence(s). Although homology can also be considered in terms of similarity (i.e. amino acid residues having similar chemical properties/functions), in the context of the present invention it is preferred to express homology in terms of sequence identity.

Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity. Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (Devereux et al 1984 Nuc. Acids Research 12 p387). Examples of other software than can perform sequence comparisons include, but are not limited to, the BLAST package (see Ausubel et al., 1999 Short Protocols in Molecular Biology, 4^{th} Ed-Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins DG & Sharp PM (1988), Gene 73(1), 237-244).

Once the software has produced an optimal alignment, it is possible to calculate % homology, preferably % sequence identity. The software typically does this as part of the sequence comparison and generates a numerical result.

The sequences may have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine hereinafter referred to as Z), diaminobutyric acid ornithine hereinafter referred to as B), norleucine ornithine (hereinafter referred to as O), pyriylalanine, thienylalanine, naphthylalanine and phenylglycine.

Replacements may also be made by unnatural amino acids. Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon RJ et al., PNAS (1992) 89(20), 9367-9371 and Horwell DC, Trends Biotechnol. (1995) 13(4), 132-134.

Preferably, if the mutation is in the coding region, then the sequences may have deletions, insertions or substitutions of amino acid residues which produce a substance that has a reduced activity or has no activity or has a different activity than the non-modified substance. Deliberate amino acid substitutions may be made on the basis of similarity in polarity, charge, solubility, hydrophobicity, hydrophilicity, and/or the amphipathic nature of the residues as long as the activity of the substance is changed.

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hybridising under high stringent conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under stringent conditions (e.g. 50° C. and 0.2×SSC).

In a more preferred aspect, the present invention covers nucleotide sequences that can hybridise to the nucleotide sequence of the present invention, or the complement thereof, under high stringent conditions (e.g. 65° C. and 0.1×SSC).

The nucleotide sequence of the present invention may be present in a vector.

The vectors for use in the present invention may be transformed into a suitable host cell.

The present invention also encompasses expression vectors comprising the sequence of the present invention.

The term “expression vector” means a construct capable of in vivo or in vitro expression.

Preferably, the expression vector is incorporated into the genome of a suitable host organism. The term “incorporated” preferably covers stable incorporation into the genome.

The choice of vector eg. a plasmid, cosmid, or phage vector will often depend on the host cell into which it is to be introduced.

The vectors for use in the present invention may contain one or more selectable marker genes.

Vectors may be used in vitro, for example for the production of RNA or used to transfect, transform, transduce or infect a host cell.

Thus, in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector.

The vector may further comprise a nucleotide sequence enabling the vector to replicate in the host cell in question.

Enhanced expression of the nucleotide sequence of the present invention may also be achieved by the selection of heterologous regulatory regions, e.g. promoter, secretion leader and terminator regions.

The present invention also encompasses constructs comprising the sequence of the present invention. The construct may even contain or express a marker, which allows for the selection of the genetic construct.

The term “host cell”—in relation to the present invention includes any cell that comprises either the nucleotide sequence or an expression vector as described above.

Thus, a further embodiment of the present invention provides host cells transformed or transfected with a nucleotide of the present invention. The cells will be chosen to be compatible with the said vector and may for example be prokaryotic (for example bacterial), fungal, yeast or plant cells. Preferably, the host cells are not human cells.

Examples of suitable bacterial host organisms are gram positive or gram negative bacterial species.

The term “organism” in relation to the present invention includes any organism that could comprise the nucleotide sequence according to the present invention and/or products obtained therefrom.

Suitable organisms may include a prokaryote, fungus, yeast or a plant. A preferred organism is a plant.

The term “transgenic organism” in relation to the present invention includes any organism that comprises the nucleotide sequence according to the present invention and/or the products obtained therefrom. Preferably the nucleotide sequence is incorporated in the genome of the organism.

The transgenic organism of the present invention includes an organism comprising any one of or combinations of, the nucleotide sequence according to the present invention, constructs according to the present invention, vectors according to the present invention, plasmids according to the present invention, cells according to the present invention, tissues according to the present invention, or the products thereof.

For example the transgenic organism may comprise a nucleotide sequence coding for a heterologous protein under the control of a mutated promoter according to the present invention.

As indicated earlier, the host organism can be a prokaryotic or a eukaryotic organism.

Teachings on the transformation of prokaryotic hosts is well documented in the art, for example see Sambrook et al (Molecular Cloning: A Laboratory Manual, 2nd edition, 1989, Cold Spring Harbor Laboratory Press). If a prokaryotic host is used then the nucleotide sequence may need to be suitably modified before transformation—such as by removal of introns.

Another host organism can be a plant. A review of the general techniques used for transforming plants may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

The promoter of the present invention may be used to express a heterologous protein. The heterologous protein may be a fusion protein.

The sequences for use according to the present invention may also be used in conjunction with one or more additional sequences—such as sequences coding for proteins of interest (POIs) or nucleotide sequences of interest (NOIs).

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### EXAMPLES

The present invention will now be described in more detail with reference to the following non-limiting examples.

The Examples refer to the accompanying figures in which:

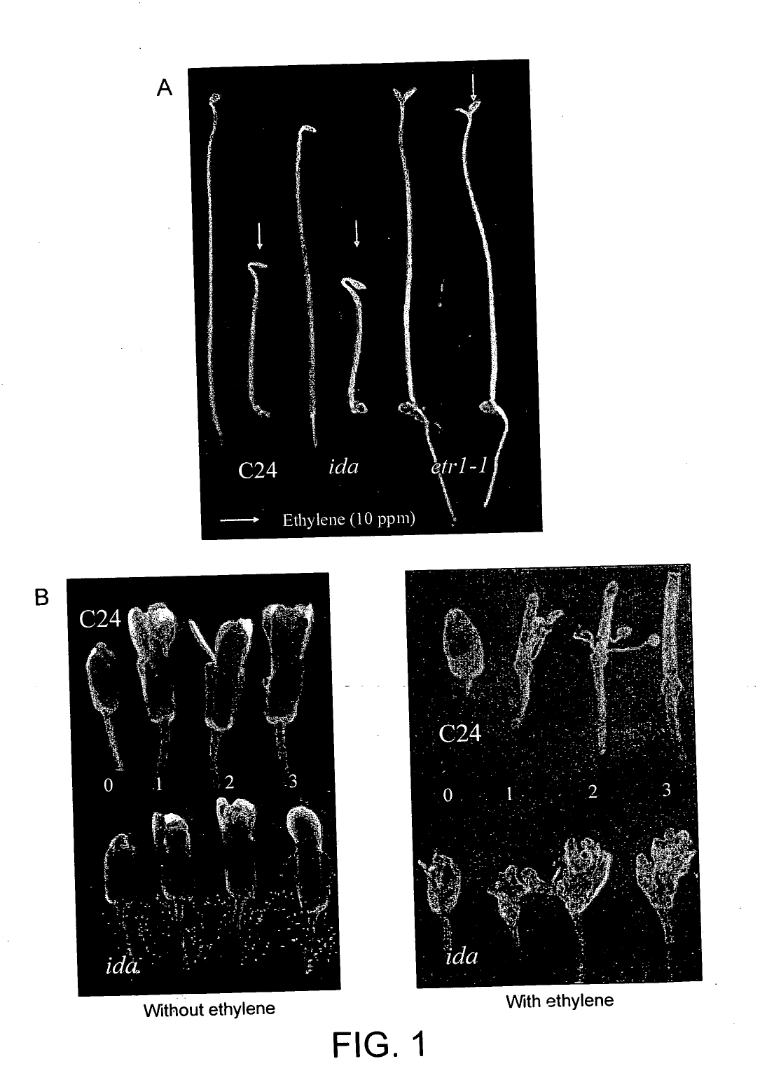

FIG. 1. Shows the ida mutant's response to ethylene.

(A) The “tripple-response” assay was conducted by exposing seedling to 10 ppm ethylene. Note that ida reacts as wt seedling, while etr1-1 is insensitive to ethylene, and behaves like a control seedling subjected to air.

(B) Comparison of flowers of position 0 to 3, exposed to air or to 10 ppm ethylene. In wt flowers exposure to ethylene causes senescence and floral organ abscission already in position 1 flowers. In contrast, ida flowers senesce, but do not abscise.

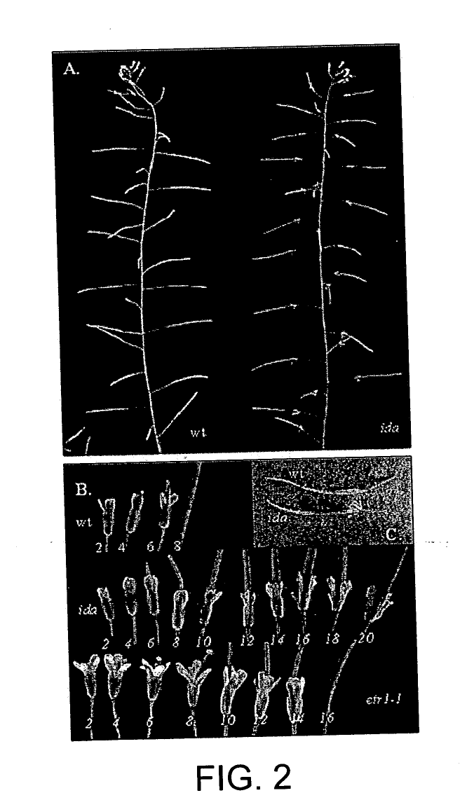

FIG. 2. Phenotype of the ida mutant compared to wt and etr1-1.

(A) Inflorescence of wt Arabidopsis plant and the ida mutant. Arrow indicates the first wt flower position where the floral organs have abscised. Note that the silique in this position is not full-grown. Note ida flowers attached along the whole inflorescence.

(B) Flowers from every second position along the inflorescence are depicted. Note indications of senescence in ida flowers from position 16, showing that the mutant is sensitive to ethylene. Note fresh, turgid and vivid floral organs in all positions prior to abscission in the ethylene-insensitive etr1-1 mutant.

(C) Septum from dry plants after dehiscence of seeds with dry floral organs still attached in the ida mutant, in contrast to wt.

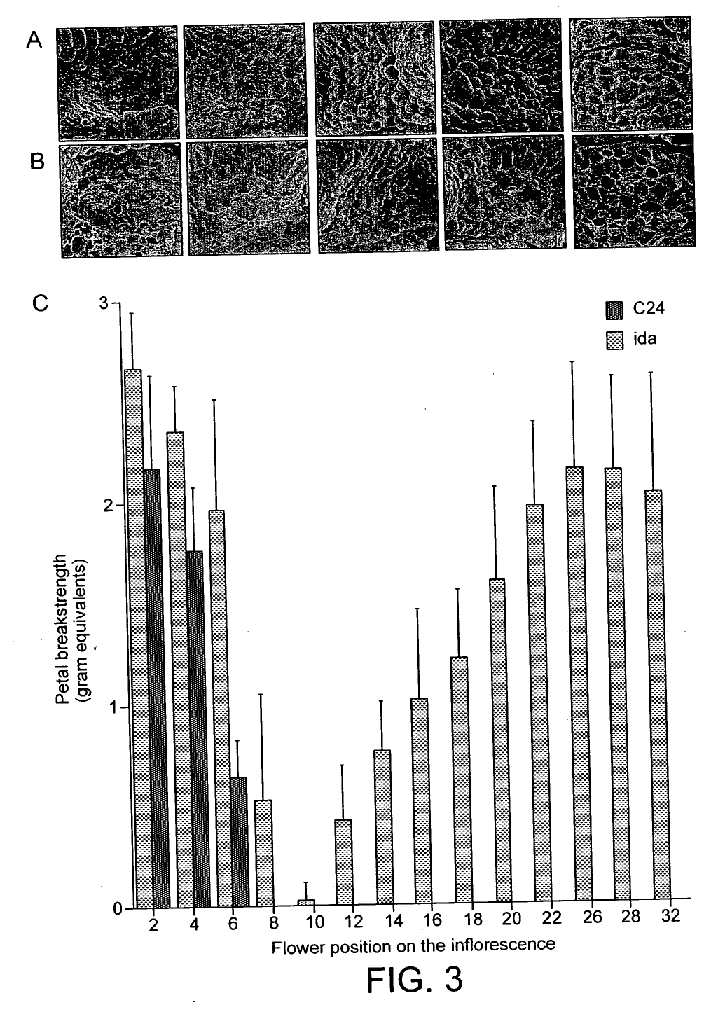

FIG. 3. Morphology of abscission zones and petal breakstrength.

Scanning electron microscopy of AZ of petals in (A) wt and (B) ida flowers at position 4 (i), 8 (ii), 10 (iii), 12 (iv) and 22 (iv) after forcible removal or natural abscission. Wt petals abscize naturally at position 8.

(C) Petal breakstrength, i.e. the force required to remove petals from flowers, measured at every second flower position on the inflorescence. Breakstrength was measured for 15 wt and ida plants, with a minimum for measurements at each position. Standard deviations are shown as thin lines on top of the columns.

FIG. 4. Identification of the IDA gene.

(A) The single T-DNA with 1239 bp of vector backbone was inserted in chromosome 1 between the genes At1g68780 and At1g68765 in the ida line. The T-DNA insertion had resulted in a 74 bp target site deletion (Δ). The distances in bp from the stop codon and the start codon of these two genes, respectively, to the T-DNA insertion point are shown, as well as the number of bp from the start to the stop codons in the two genes. The single intron in At1g68780 is indicated by a darker color. Direction of transcription is indicated by arrows. The extension of the two genomic fragments used in a complementation experiment is shown below the genomic region. All elements in the drawing are not to scale.

(B) The phenotype of ida plants transformed with the 2633 bp fragment encompassing the At1g68780 gene (with 959 bp of upstream and 154 bp of downstream sequence) was identical to the ida mutant phenotype. Note flowers attached to full-grown siliques.

(C) The phenotype of ida plants transformed with the 2019 bp fragment (starting 8 bp downstream of the stop codon of the At1g68780 gene and ending 303 bp downstream of the At1g68765 open reading frame) was identical to wt phenotype. Note that floral organs are only seen in the upper positions of the inflorescence.

(D) RT-PCR on mRNA from position 1-8 flowers, amplified the expected 421 bp fragment with IDA primers from wt, but not from and ida plants, while a 294 bp ACTIN2-7 fragment was amplified from both wt and ida template. M-φX174 digested with Hae III.

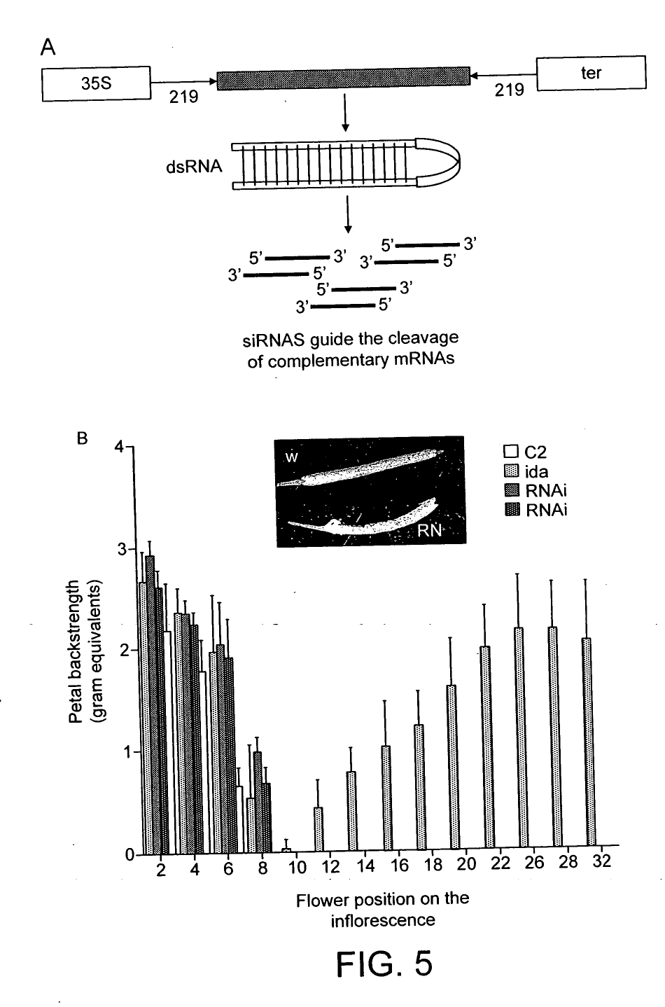

FIG. 5. RNA interference.

(A) Construct used to transform wt Arabidopsis plants with 219 bp of the IDA open reading frame cloned in inverse orientation on each side of a GUS stuffer fragment. Transcription from the 35S promoter will generate an mRNA that can form double stranded RNA (dsRNA) with the GUS part as a loop. dsRNA will be degraded to small interfering RNAs (siRNA) that will mediate degradation of the normal IDA mRNA. ter-terminator.

(B) Breakstrength of petals of two RNAi plants compared to wt and the ida mutant at every second position along the inflorescence. The RNAi plants have the same profile as ida until to position 10 and display delayed abscission compared to wt.

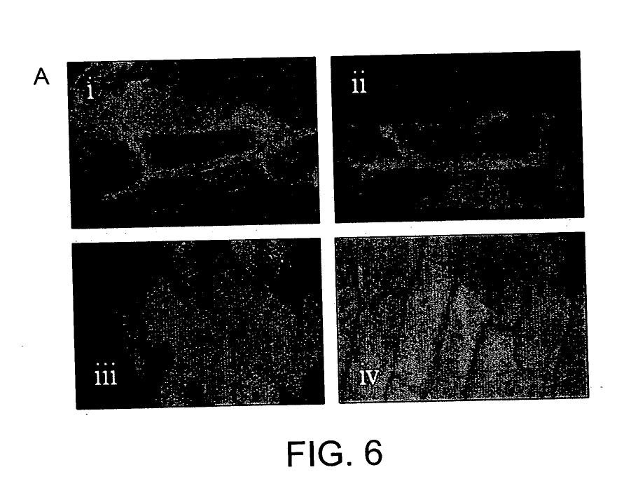

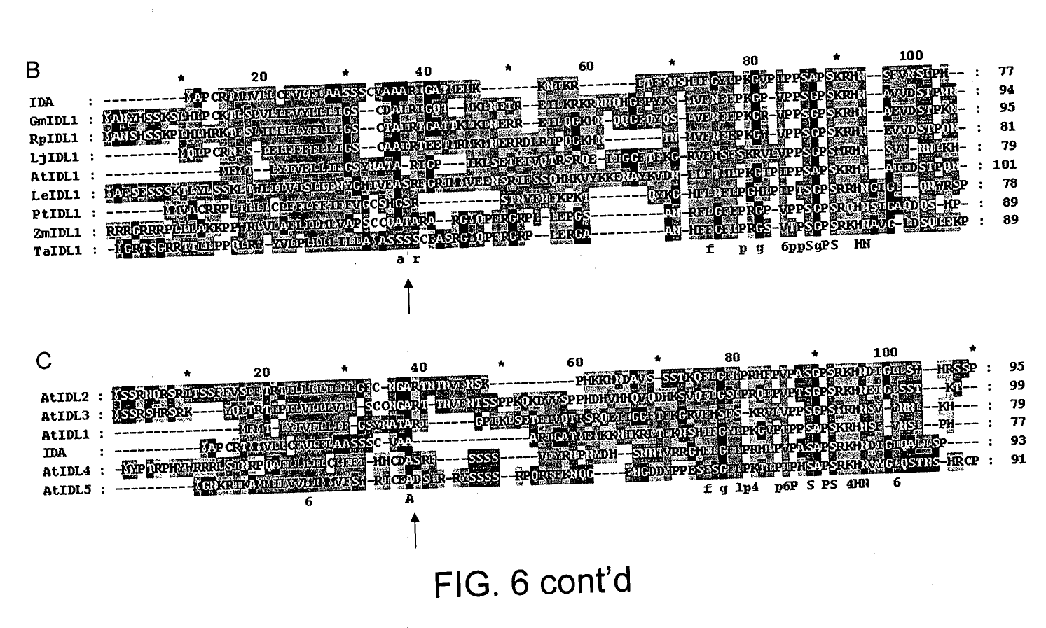

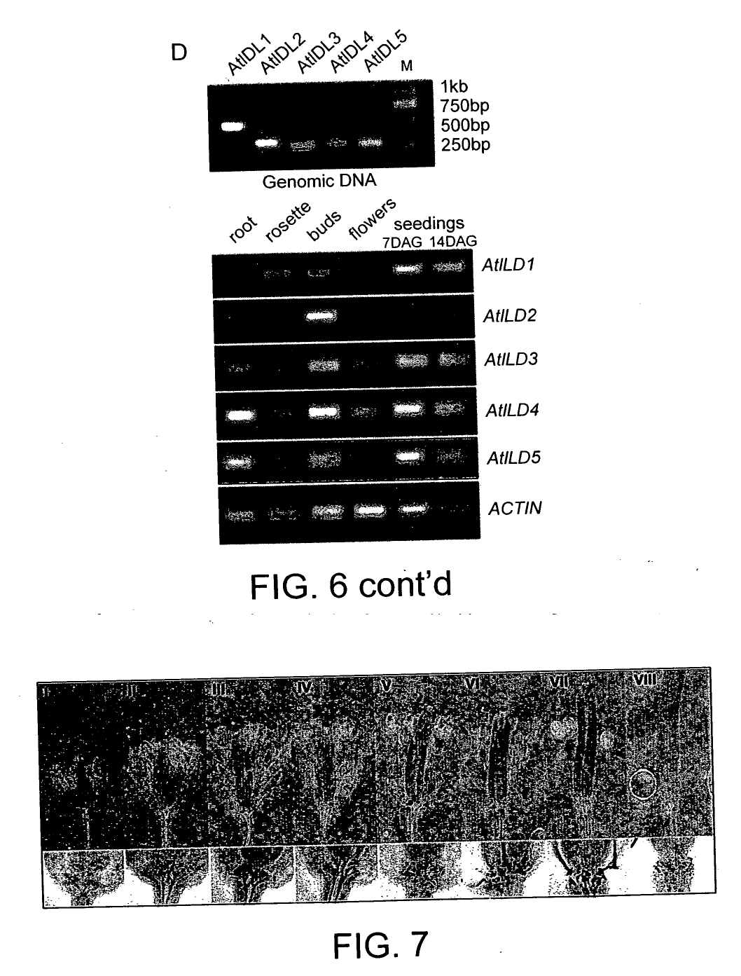

FIG. 6 Features of the IDA protein.

A Subcellular localization of proteins in onion epidermis transient expression assay. Extra-cellular localization of (i) IDA-GFP fusion protein and (ii) IDA-signal peptide-GFP-fusion protein. (iii) Cytoplasmic localization of GFP alone. (iv) Nuclear localzation of HP1-GFP fusion protein in the same assay. GFP fluorescence was revealed 2 h after bombardment utilizing a microscope equipped with epifluorescence and Normarski optics.

B. Alignment of IDA and IDA-like (IDL) proteins encoded by cDNAs from Arabidopsis (AtIDL1, gene At3g25655), tomato (LeIDL1, ac. no AI779570); lotus (LjIDL1, ac. NO. AW719486), soybean (GnIDL1, ac.NO. Bq630646), black locust (RpIDL1, ac.NO. BI642538), maize (ZmIDL1, ac.NO. BI430572), poplar (PtIDL1, ac.NO. BU889756), and wheat (TaIDL1, acno BM135459). The first 20 aa of ZmIDL1 are not shown. Note the hydrophobic predicted signal peptides, and the arrow indicating the position of the predicted cleavage sites.

C. Alignment of Arabidopsis IDL proteins. Note the hydrophobic predicted signal peptide. The arrow indicates the predicted cleavage sites, in all proteins after an alanine residue. The genes AtIDL2-5 were identified by using the C-terminal 20 aa of the IDA protein in a tBLASTn search against the Arabidopsis genome. The hits found were inspected for short open reading frames encoding proteins with a predicted signal peptide (using SignalP) and similarity to IDA in the C-terminal end. Amino acids are shaded according to properties: Yellow—hydrophobic residues; grey—basic residues; green—small residues; blue—polar non-aliphatic residues.

D RT-PCR on mRNA from different tissues, as indicated (lower panel), using primers amplifying fragments of 465 bp (AtIDL1), 280 bp (AtIDL2), 256 bp (AtIDL3), 261 bp (AtIDL4) and 259 bp (AtIDL5), cfr. upper panel where genomic DNA was used as template and the marker line (M) is shown. Note that the positive control ATCIN2-7 was amplified at comparable levels from all tissues. DAG—days after germination.

FIG. 7. Abscission zone specific expression directed by the IDA promoter.

Marker gene (gus encoding β-glucoronidase) expression directed by the IDA promoter to the abscission zone both at the plant body side and in the abscising organs. Marker gene expression is evident from floral position five (V) to eight (VIII). The floral organs have abscised at position eight in wild type flowers. Flowers were stained for GUS-activity by immersing them in X-gluc solution (1 mg/ml X-glucA in 0.01 M Na₂PO₄ pH 7, 0.5% Trition X-100). Samples were incubated at 37° C. over night. Chlorophyll was removed by washing three times 30 min with Abs EtOH:Acetic acid (1:1). Stained tissues were pictured through a Leica WILD Mz8 binocular using a Nikon COOLPIX 995 digital camera. Using in situ hybridization on sections of Arabidopsis flowers, confirmatory results are obtained using the promoter-reporter gene construct—i.e. that the IDA gene is expressed in abscission zones.

In screening for mutants delayed in floral abscission, the mutant inflorescence deficient in abscission, ida, was identified in a collection of Arabidopsis lines transformed with the transfer-DNA (T-DNA) vector pMHA2 (10). In contrast to etr1 and ein2, the ida mutant is sensitive to ethylene (FIG. 1). The ‘triple-response’ assay has been used to identify mutants altered in ethylene synthesis, perception and responses (11). Seedlings of the ida mutant germinated on the natural precursor of ethylene, 1-aminocyclopropane-1-carboxylic acid (ACC) (not shown), or exposed to 10 ppm ethylene (FIG. 1A), displayed the same drastic morphological changes characteristic of wt seedlings, i.e. inhibition of root and hypocotyl elongation, radial swelling of the hypocotyl and root, and exaggeration in the curvature of the apical hook. Thus, in contrast to etr1-1 seedlings, ida seedlings seem perfectly capable of perceiving and responding to ethylene. Incubation of plants in chambers with 10 ppm ethylene, results in wilting of rosettes and cauline leaves in wt and ida plants, only etr1-1 plants are unaffected by the treatment (not shown). In wt plants this level of ethylene also results in senescence and abscission floral organs shortly after the opening of the flower. In the ida plants, floral organs senesce, but do not abscise (FIG. 1B).

In wt Arabidopsis flowers, the shedding of turgid flower petals, sepals and stamens, supervenes shortly after anthesis (FIG. 2) (1). In contrast, the mutant ida retain these floral parts indeterminately (past position 30) (FIG. 2A). At position 2, 4 and 6 (counted from the first flower with visible white petals at the top of the inflorescence) the organs of wt, etr1-1 and ida are turgid and attached to the developing silique (FIG. 2B). At position 8 the perianth and stamens abscise from the wt flower. The floral organs of the ida mutant are still retained at position 16, when the etr1-1 mutant has discarded its flowers (FIG. 2B).

After the shedding of mature seeds the now completely dry, colorless and transparent floral parts of the mutant ida are still attached (FIG. 2C). The opening and dehiscence of the valves and the discarding of seeds from the false septum of ida strongly indicates the mutation to be involved in a pathway specific in controlling floral abscission.

The mutants etr1 (7,12) and ein1 (8,13), show some similarity to the ida mutant. However, in addition to the aberrant pattern of floral abscission discussed above, these mutants show delayed leaf senescence, larger rosettes, a delay in bolting and flowering, and very low seed germination rates, when compared to wild-type plants (7,8,14). In the ida mutant, no developmental processes other than floral abscission was observed to be affected.

In Arabidopsis the floral AZ develops as a band of small, densely cytoplasmic cells at the junction between the flower receptacle and floral organs. Scanning electron microscopy was used to compare the morphology of wt petal AZ to that of ida (FIGS. 3A and B) after either forcible removal or natural abscission of the petals. A flattened fracture plane is observed shortly after anthesis in wt and in the ida mutant, when petals have been removed. Broken cells are revealed at this stage (FIG. 3A, i and 3B, i). A flattened fracture plane is observed at position 8 (FIG. 3A, ii and 3B, ii), which corresponds to the time when the floral organ abscise in the wt. At position 10, the wt fracture plane cells have started to attain a rounded appearance (FIG. 3A, iii), while the ida fracture plane still looks like a flattened cavity (FIG. 2B, ii and iii). By position 12 and onwards, wt plants show fully rounded cells as a protective scar layer is formed (FIG. 2A, iv and v). In the mutant forcible removal of petals shows uneven rounding of AZ cells (FIG. 2B, iv), and thereafter an increase in the number of broken cells with a strong similarity to those seen in early positions (FIG. 2B, v). This indicates the breaking of primary cell walls.

To obtain a quantitative evaluation of the difference between wt and ida, the force needed to remove petals from the plant was measured using a stress transducer (5). As shown in FIG. 3C, the breakstrength of wt petals decrease rapidly from position zero. At position 8 the breakstrength is reduced to nil. The ida mutant initially show a similar, but delayed, breakstrength profile and approaches zero at position 10. However, from position 12 the breakstrength increases again, so that the oldest flowers on an inflorescence position 32) has a breakstrength similar to the youngest flowers. This, in addition to the SEM data, indicate that the initial steps in the abscission process are only delayed in the ida mutant, while the major effect of the mutation is seen at the stage when separation of the floral organ from the main body of the plant normally would take place.

To separate the floral organ form the plant body, the shared cell wall or middle lamella has to be dissolved. A number of cell wall degrading enzymes, most notably polygalaturonases (PGs) and β-1,4-endo-glycanases (EGases) that are represented by large gene families, are important in this process (15), but very few genes have been reported to be specific for abscission. In tomato, the JOINTLESS gene encoding a MADS-box transcription factor controls the formation of the AZ of the pedicel (16). JOINTLESS and AGL15 appoint MADS domain proteins as important in the abscission process. However, the unique phenotype of the ida mutant suggested the involvement of a yet uncharacterised gene.

To identify the genetic basis for the ida phenotype, the mutant was crossed to wt Arabidopsis. F1 plants from the cross did not show the phenotype. In the F₂ generation the number of wild-type and ida plants were consistent with a 3:1 ratio (37 wt: 11 ida, χ²=0.13, P>0.7), which would be expected if the ida phenotype is the manifestation of a homozygous recessive, monogenic mutation. All progeny plants displaying the ida phenotype were homozygous for the single T-DNA present in the ida mutant line (17). The co-segregation of homozygousity of the T-DNA and the ida phenotype, suggested that the T-DNA insertion was causing the mutant phenotype.

Cloning of the plant DNA flanking the T-DNA (18) revealed that the T-DNA was inserted in chromosome 1, between an annotaded gene encoding a putative protein with ten LRRs (At1g68780), and a small, intronless gene (At1g68765) (FIG. 4A). To investigate whether any of these genes were affected by the T-DNA insertion, we transformed ida mutant plants with two genomic fragments (19), one covering the LRR gene, and another covering the small open reading frame with upstream and downstream sequences (FIG. 4A). 59 and 58 independent transformants were generated for each construct, respectively. All the transformants harbouring the LRR gene displayed the mutant ida phenotype (FIG. 4B), while 54 plants transformed with the other construct all showed a wild type abscission pattern (FIG. 4C). Since the small open reading frame with its upstream and downstream sequences can complement the mutant phenotype, we conclude that we have identified the IDA gene (20). This also means that the DN fragment used for complementation contains a functional IDA promoter.

To investigate the expression pattern of the wt IDA gene, a promoter fragment of 1419 bp was amplified by PCR with the primers 5′ TTT TCA ATT TTG TRA TTG CAT 3′ and 5′ ATT TGG TAG TCA ATG TTT TTT TTC 3′ (cf ID sequence I) and inserted in the Sma I site of the pPZP211G vector generating the construct pPZP IDA::GUS. The pPZP211 G vector is a pPZP vector (Hajdukiewicz et al., Plant Mol. Biol. 989-994, 1994) which between the T-DNA right and left borders carries the nptII gene as a plant selectable marker, and a promoterless GUS gene with a nos terminator inserted between the Sma I and EcoR I sites of the polylinker. Transformed Arabidopsis plants were investigated for marker gene expression. Expression was observed in the abscission zone on the plant body side from position 5 to 8, and at the base of the petals from position 5 and until they abscised (FIG. 7).

FIG. 7 presents the abscission zone specific expression directed by the IDA promoter. Marker gene (gus encoding β-glucoronidase) expression directed by the IDA promoter to the abscission zone both at the plant body side and in the abscising organs. Marker gene expression is evident from floral position five (V) to eight (VIII). The floral organs have abscised at position eight in wild type flowers. Flowers were stained for GUS-activity by immersing them in X-gluc solution (1 mg/ml X-glucA in 0.01 M Na₂PO₄ pH 7, 0.5% Trition X-100). Samples were incubated at 37° C. over night. Chlorophyll was removed by washing three times 30 min with Abs EtOH:Acetic acid (1:1). Stained tissues were pictured through a Leica WILD MZ8 binocular using a Nikon COOLPIX 995 digital camera.

A cDNA corresponding to this gene with 98 bp 5′ UTR and a 205 bp 3′ UTR, has recently been cloned (ac.NO. AY087883). The T-DNA in the ida mutant therefore seems to be positioned in the promoter of the IDA gene. RT-PCR on mRNA of flowers (position 1 to 8), amplified the expected PCR fragment from wild type but not from mutant tissue (FIG. 3D) (21). This experiment suggests that the T-DNA insertion has interfered with normal gene expression.

To further confirm that the manipulation of the expression level of the IDA gene will affect abscission, Arabidopsis plants were transformed with a construct designed for RNA interference (RNAi) (22). A DNA fragment encompassing the open reading frame of the IDA gene was cloned on each side of a fragment of the GUS reporter gene, in inverse orientation, in a T-DNA vector (FIG. 5A). The 35S promoter from Cauliflower mosaic virus (CaMV) will drive expression of a double-stranded RNA (dsRNA), that can interfere with normal IDA expression. Selected transformant were subjected to breakstrength measurements. Two transformants with delayed abscission are shown in FIG. 5B. Both have the same break-strength profile as the ida mutant down to position ten, and retain their floral organs significantly longer than wt plants. However, an increase in break-strength from position 10 until plant maturity was not seen in the RNAi plants. We assume that more efficient interference with IDA expression can be achieved using vectors like pKANNIBAL and pHELLSGATE where an intron is included as a stuffer fragment between the inverted gene fragments to improve the formation of dsRNA (23). Another possibility is to useIDA's own promoter to drive dsRNA expression.

The IDA gene encodes a small protein of 77 amino acids (aa) with a N-terminal hydrophobic region predicted with SignalP to act as a signal peptide (24). To investigate whether the IDA protein is exported, onion cells were bombarded with constructs from which the IDA cDNA or the putative signal peptide would be expressed in fusion with the Green Fluorescent Protein (GFP) (25). Both the IDA-GFP (FIG. 6A, i) and the signal peptide-GFP fusion proteins (FIG. 6A, ii) are localized extra-cellularly. In contrast, GFP alone is expressed in the cytoplasm (FIG. 6A, iii), while a fusion to the Drosophila heterochromatin protein 1 (HP1), directs GFP to the nucleus (FIG. 6A, iv).

The likely localization of the IDA protein to the extra-cellular space, its small size and high pI (11.87) are features similar to the ligand CLAVATA3 (CLV3) (26), required to hinder overproduction of flowers and floral organs. A large familiy of CLV3-like (CLE) genes have recently been identified by iterative BLAST searches in the Arabidopsis database (27). Likewise, a family of putative ligands with similarity to the protein encoded by the S-locus Cystein-rich (SCR) gene involved in incompatibility in Brassica (28) has recently been found. The CLE and the SCR-Like (SCRL) proteins are suggested to represent ligands for RLKs. In the Arabidopsis genome, there are more than 400 RLKs that can be divided into more than 21 classes based on their extracellular domains (29).

Without wishing to be bound by theory, the inventors believe that the IDA protein represents a new class of ligands in plants. A BLASTP search identified further IDA-LIKE (IDL) cDNAs, in other plant species including Arabidopsis (12.24), lotus (11.13), tomato (11.02), soybean (11.74), black locust (11.42), maize (12.62), poplar (12.53), and wheat (11.37) that can encode similar short proteins with hydrophobic N-terminals, isoelectric points close to that and a conserved C-terminal motif (v/iPPSa/gPSk/rk/rHN) (FIG. 6B) which is distinct from the CLE-motif and the cysteine-rich pattern of SCRLs. BLASTP searches against the translated Arabidopsis genome, and RT-PCR analysis show the presence of four additional IDL genes encoding proteins of less than 100 aa (see FIGS. 6C and D, and Table 1). Genes of such small size are often overlooked by automated annotation programs. Given the common features of different cell separation processes in plants (15), it is currently believed that IDL proteins serve functions in other abscission processes, like dehiscence of seeds and fruits, or abscission of leaves.

TABLE 1
Features of IDL genes and cDNAs, and their encoded proteins.

| | | | | | | Tissue source of |
|---|---|---|---|---|---|---|
| Protein | Protein ID/ | Gene/ | BAC clone | | | cDNAs/ |
| name | cDNA Ac. no | BAC clone | position | Chr^{a} | pI^{b} | expreesion pattern |
| IDA | AAM65435.1 | At1g68765 | | I | 11.87 | See FIG. 6, 7 |
| AtIDL1 | AAM63318.1 | At3g25655 | | III | 12.24 | top-most |
| | | | | | | inflorescence tissues |
| | | | | | | and roots |
| AtIDL2 | | MUB3 | 69965-70249 | V | 12.52 | See FIG. 6D |
| AtIDL3 | | F17I14 | 1503-1207 | V | 10.61 | See FIG. 6D |
| AtIDL4 | | MVE11 | 24901-25179 | III | 11.92 | See FIG. 6D |
| AtIDL5 | | F22K20 | 33903-33595 | I | 10.18 | See FIG. 6D |
| GmIDL1 | BQ630646 | | | | 11.74 | Roots |
| LeIDL1 | AI779570 | | | | 11.02 | Pseudomonas |
| | | | | | | susceptible tomato |
| LjIDL1 | AW719486 | | | | 11.13 | Nodules |
| PtIDL1 | BU889756 | | | | 12.53 | Petioles |
| RpIDL1 | BI642538 | | | | 11.42 | sapwood/heartwood |
| | | | | | | transition zone |
| TaIDL1 | BM135459 | | | | 11.37 | Fusarium |
| | | | | | | graminearum |
| | | | | | | infected spikes |
| ZmIDL1 | BI430572 | | | | 12.62 | juvenile vegetative |
| | | | | | | shoots |

^{a}Arabidopsis chromosome.
^{b}Isoelectric point of proteins without putative N-terminal signal peptides, as calculated by Isoelectric⁺ of the Genetics Computer Group, inc.

The ida mutant phenotype and the characteristics of the IDA protein make it possible to refine a working model for the abscission process (4). First the AZ with small dense cells is formed at the base of the organ to be shed. Obviously, mutants deficient in the formation of an AZ, like the jointless mutant in tomato, cannot go through abscission (16). However, the ida mutant demonstrates that the development of an AZ is not sufficient for abscission to take place. In the second stage of abscission, the AZ is presumed to acquire the competence to respond to abscission signals. In responsive AZs, ethylene can speed up the activation of the abscission process. However, the ethylene-sensitive ida mutant shows that ethylene in itself is not sufficient for abscission to take place. The abscission process involves a maturations stage with changes in cell extensibility and elongation, as well as the actual dissolution of the middle lamellae between the cells on the main body of the plant and the organ to be shed. The initial decrease in petal breakstrength and tendencies of rounding of cells in the AZ (position 12) of the ida mutant, indicates that these aspects of AZ maturation are not sufficient for abscission to take place: The genuine separation step is under independent control.

The inventors believe that the IDA protein is a ligand of a receptor and that the action of the IDA gene is triggered by the culmination of AZ maturation. Without a functional IDA gene, the final separating stage of the abscission process, cell wall degradation, will not take place.

As an example of a mutation in the coding region (see SEQ ID No. 1, 2 and 12), reference may be made to the T-DNA insertion line (no. SALK_{—}133209) from the SALK collection (see http://signal.salk.edu/cpi-bin/tdnaexpress). This has the T-DNA inserted in the coding sequence of the IDA gene. Plants homozygous for this insertion show the ida mutant phenotype. The position of this T-DNA insertion is presented in SEQ ID No. Sequence 11.

### SUMMARY PARAGRAPHS

Some aspects of the present invention will now be described by way of numbered paragraphs.

1. A modified plant comprising a nucleotide sequence as shown in ID Seq. 1 or a variant, homologue, fragment or derivative thereof, wherein said sequence or the expression thereof has been modified so that the plant exhibits decreased organ abscission.

2. A plant as defined in paragraph 1 wherein the nucleotide sequence is selected from AtIDL1, gene At3g25655, tomato (LeIDL1, ac. no AI779570, lotus (LjIDL1, ac. NO. AW719486), soybean (GmIDL1, ac.NO. Bq630646), black locust (RpIDL1, ac.NO. BI642538), maize (ZmIDL1, ac.NO. BI430572), poplar (PtIDL1, ac.NO. BU889756), and wheat TaIDL1, ac.no BM135459).

3. A plant as defined in paragraph 1 or 2 wherein the nucleotide sequence includes a coding sequence for a C terminal motif PpSa/gPSk/rk/rHN.

4. A plant as defined in paragraph 1, 2 or 3 wherein the nucleotide sequence includes a coding sequence for a N terminal hydrophobic signal peptide.

5. A plant as defined in paragraphs 1 or 2 wherein the nucleotide sequence includes a coding sequence for a C terminal motif v/iPpSa/gPSK/rk/rHN and a coding sequence for an N terminal hydrophobic signal peptide.

6. A plant as defined in any of paragraphs 1 to 5 wherein the sequence is ID seq 2 [AY087883].

7. A plant as defined in any preceding paragraph wherein the decreased organ abscission is or relates to a flower or parts thereof.

8. A plant as defined in any preceding paragraph which is a flowering plant or tree.

9. A plant as defined in any preceding paragraph which is Arabidopsis thaliana.

10. A flowering plant as defined in paragraph 8 which is a crocus, tulip, cyclamen, poinsettia, lotus or rose or a tree which is a poplar or Christmas tree.

11. Seeds of other propagating material from a plant as defined in any preceding paragraph.

12. A flower from a plant according to any of paragraphs 1 to 10.

13. A process of preventing organ loss in a plant comprising modifying the sequence or expression of a sequence as defined in any of paragraphs 1 to 6.

14. A process as defined in paragraph 13 wherein the modification of the nucleotide sequence is by mutation or deletion.

15. A process as defined in paragraph 13 wherein the modification is of a promoter or other regulatory sequence.

16. A method according to paragraph 13 wherein the modification is achieved by the use of an antisense construct or a RNA interference construct.

17. An isolated nucleotide sequence comprising the sequence as shown in ID Seq. 1 or a variant, homologue, fragment or derivative thereof.

18. A sequence as defined in paragraph 17 which is (AtIDL1, gene At3g25655), tomato (LeIDL1, ac. no AI779570), lotus (LjIDL1, ac. NO. AW719486), soybean (GMIDL1, ac.NO. Bq630646), black locust (RpIDL1, ac.NO. BI642538), maize (ZmIDL1, ac.NO. BI430572), poplar (PtIDL1, ac.NO. BU889756), and wheat (TaIDL1, ac.no BM135459).

19. A sequence as defined in paragraph 17 or 18 which is ID seq. 2 [AY087883]

20. A nucleotide sequence which is antisense to a nucleotide sequence as defined in any one if paragraphs 17 to 19.

21. A sequence according to paragraph 17 which is a promoter or other regulatory sequence.

22. An isolated amino acid sequence comprising the sequence as shown in ID Seq. 3 or a sequence substantially homologous thereto, or a fragment thereof.

23. A vector comprising a nucleotide sequence as defined in any one of paragraphs 17 to 21.

24. A host cell transfected or transformed with a nucleotide sequence as defined in any one of paragraphs 17 to 21.

25. The use of an isolated nucleotide sequence as defined in any one of paragraphs 17 to 21, in the control of plant organ abscission.

26. A method of decreasing organ abscission in a plant, comprising reducing the expression level of a gene in a plant, wherein the gene is the IDA gene or a mimetic thereof.

27. A method of decreasing organ abscission in a plant, comprising reducing the expression level of a gene in a plant, wherein the gene includes a coding sequence for a C terminal motif PpSa/gPSk/rk/rHN.

28. A method of decreasing organ abscission in a plant, comprising reducing the expression level of a gene in a plant, wherein the gene includes a coding sequence for a N terminal hydrophobic signal peptide.

29. A method of decreasing organ abscission in a plant, comprising reducing the expression level of a gene in a plant, wherein the gene includes a coding sequence for a C terminal motif v/iPpSa/gPSK/rk/rHN and a coding sequence for an N terminal hydrophobic signal peptide.

30. A method of decreasing organ abscission in a plant, comprising reducing the expression level of a gene in a plant, wherein the gene is selected from:. AY087883, AtIDL1, gene At3g25655, tomato (LeIDL1, ac. no AI779570, lotus (LjIDL1, ac. NO. AW719486), soybean (GmIDL1 ac. NO. Bq630646), black locust (RpIDL1, ac. NO. BI642538), maize (ZmIDL1, ac. NO. BI430572), poplar (PtIDL1, ac. NO. BU889756), and wheat (TaIDL1, ac. no BM135459).

31. A method of decreasing organ abscission in a plant, comprising reducing the expression level of a gene in a plant, wherein the gene is or comprises all or part of the nucleotide sequence presented as any one of SEQ ID No. 1, SEQ ID No. 2, SEQ ID No. 8, SEQ ID No. 9, SEQ ID No. 10 or SEQ ID No. 11 or a variant of any thereof or a homologue of any thereof.

32. A method of decreasing organ abscission in a plant, comprising reducing the expression level of a gene in a plant, wherein the gene is or comprises all or part of the nucleotide sequence presented as any one of SEQ ID No. 1, SEQ ID No. 2, SEQ ID No. 8, SEQ ID No. 9, SEQ ID No. 10 or SEQ ID No. 11.

33. A method according to any one of the preceding paragraphs wherein said expression level is reduced by mutating said gene.

34. A method according to any one of the preceding paragraphs wherein said expression level is reduced by mutating a regulatory region of said gene.

35. A method according to any one of the preceding paragraphs wherein said expression level is reduced by mutating the promoter region of said gene.

36. A method according to any one of the preceding paragraphs wherein said expression level is reduced by mutating the promoter region of said gene by use of T-DNA insertion techniques or by use of interfering moieties.

37. A nucleotide sequence; wherein the nucleotide sequence is a modified sequence;
wherein when a plant comprises or is transformed with said modified nucleotide sequence said plant exhibits decreased organ abscission.

38. A nucleotide sequence; wherein the nucleotide sequence is all or part of a sequence selected from: AY087883, AtIDL1, geneAt3g25655, tomato (LeIDL1, ac. No. AI779570, lotus (LjIDL1, ac. NO. AW719486), soybean (GmIDL1, ac. NO. Bq630646), black locust (RpIDL1, ac. NO. BI642538), maize (ZmIDL1, ac. NO. BI430572), poplar (PtIDL1, ac. NO. BU889756), and wheat (TaIDL1, ac.no BM135459), a nucleotide sequence that includes a coding sequence for a C terminal motif PpSa/gPSk/rk/rHN, a nucleotide sequence that includes a coding sequence for an N terminal hydrophobic signal peptide, a nucleotide sequence that includes a coding sequence for a C terminal motif v/iPpSa/gPSK/rk/rHN and a coding sequence for an N terminal hydrophobic signal peptide; wherein said nucleotide sequence comprises one or more mutations in any of said sequence; and wherein when a plant comprises or is transformed with said nucleotide sequence said plant exhibits decreased organ abscission.

39. A nucleotide sequence according to paragraph 37 or paragraph 38 wherein said sequence is any one of SEQ ID No. 1, SEQ ID No. 2, SEQ ID No. 8, SEQ ID No. 9, SEQ ID No. 10 or SEQ ID No. 11 or a variant of any thereof or a homologue of any thereof or a mimetic of any thereof; and wherein said nucleotide sequence comprises one or more mutations in any of said sequence.

40. A nucleotide sequence according to any one of paragraphs 37 to 39 wherein said sequence is any one of SEQ ID No. 1, SEQ ID No. 2, SEQ ID No. 8, SEQ ID No. 9, SEQ ID No. 10 or SEQ ID No. 11; and wherein said nucleotide sequence comprises one or more mutations in any of said sequence.

41. A nucleotide sequence that is a mutation of the sequence shown as any one of SEQ ID No. 1, SEQ ID No. 2, SEQ ID No. 8, SEQ ID No. 9, SEQ ID No. 10 or SEQ ID No. 11 or a mimetic of any thereof; wherein when a plant comprises or is transformed with said nucleotide sequence said plant exhibits decreased organ abscission.

42. A nucleotide sequence that is a mutation of the sequence shown as any one of SEQ ID No. 1, SEQ ID No. 2, SEQ ID No. 8, SEQ ID No. 9, SEQ ID No. 10 or SEQ ID No. 11; wherein when a plant comprises or is transformed with said nucleotide sequence said plant exhibits decreased organ abscission.

43. A construct comprising the invention according to any one of paragraphs 37 to 42.

44. A vector comprising the invention according to any one of paragraphs 37 to 43.

45. An expression vector comprising the invention according to any one of paragraphs 37 to 44.

46. A transformation vector comprising the invention according to any one of paragraphs 37 to 45.

47. A host cell comprising the invention according to any one of paragraphs 37 to 46.

48. An organ comprising the invention according to any one of paragraphs 37 to 47.

49. An organism comprising the invention according to any one of paragraphs 37 to 48.

50. A transformed plant comprising the invention according to any one of paragraphs 37 to 49.

51. A transformed plant according to paragraph 50 wherein said plant is a flowering plant or tree.

52. A method comprising transforming a plant with the invention according to any one of paragraphs 37 to 46.

53. Use of the invention according to any one of paragraphs 37 to 46 in the preparation of a transformed plant to decrease organ abscission.

54. A process for decreasing organ abscission in plants by modifying the IDA gene, a homologue, fragment, or derivative thereof, or the expression thereof.

55. A nucleotide sequence, wherein when a plant comprises or is transformed with said nucleotide sequence said plant exhibits decreased organ abscission, preferably wherein said nucleotide sequence is a mutation.

56. Use of a promoter sequence comprising all or part of the nucleotide sequence presented as SEQ ID No. 1, SEQ ID No. 2, SEQ ID No. 8, SEQ ID No. 9, SEQ ID No. 10 or SEQ ID No. 11 to express an heterologous coding sequence.

57. Use according to paragraph 56 wherein said heterologous coding sequence is a mutated coding sequence corresponding to the coding sequence naturally associated with said promoter sequence.

58. An isolated and/or purified nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

59. An isolated and/or purified construct comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

60. An isolated and/or purified vector comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

61. An isolated and/or purified expression vector comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

62. An isolated and/or purified transformation vector comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11.

63. An isolated and/or purified transformed cell comprising a nucleotide sequence comprising all or part of the nucleotide sequence presented as SEQ ID No. 1, SEQ ID No. 2, SEQ ID No. 8, SEQ ID No. 9, SEQ ID No. 10 or SEQ ID No. 11; wherein said cell is not a naturally occuring cell.

64. An isolated and/or purified nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

65. An isolated and/or purified construct comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ. ID No. 11.

66. An isolated and/or purified vector comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

67. An isolated and/or purified expression vector comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

68. An isolated and/or purified transformation vector comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

69. An isolated and/or purified transformed cell comprising a nucleotide sequence that can hybridise to or is complementary to all or part of the nucleotide sequence presented as SEQ ID No. 1, SEQ ID No. 2, SEQ ID No. 8, SEQ ID No. 9, SEQ ID No. 10 or SEQ ID No. 11.

70. An isolated amino acid sequence comprising the sequence as shown in SEQ ID No. 3 or a sequence substantially homologous thereto, or a fragment thereof.

71. Use of the invention according to any one of paragraphs 37 to 46 in the preparation of a transformed plant to prevent or reduce plant cell degradation.

72. A modified plant substantially as described herein and with reference to any one of the Figures.

73. A method of preventing organ loss in a plant substantially as described herein and with reference to any one of the Figures.

74. An isolated nucleotide sequence substantially as described herein and with reference to any one of the Figures.

75. An isolated amino acid sequence substantially as described herein and with reference to any one of the Figures.

All publications mentioned herein are incorporated by reference. Various modifications and variations of the described methods and system of the present invention will be apparent to those skilled in the art without departing from the scope and spirit of the invention. Although the present invention has been described in connection with specific preferred embodiments, it should be understood that the invention as claimed should not be unduly limited to such specific embodiments. Indeed, various modifications of the described modes for carrying out the invention which are obvious to the skilled person are intended to be within the scope of the following claims.

### References and Notes

1. Bleecker, A. and Patterson, S. E. (1997) Last exit: senescence, abscission, and meristem arrest in Arabidopsis. Plant Cell 9, 1169-1179.
2. Addicott, F. T. (1982) Abscission. University of California Press, London.
3. Sexton, R. and Roberts, J. A. (1982) Cell biology of abscission. Ann. Rev. Plant Physiol., 33, 133-162.
4. Patterson, S. E. (2001) Cutting loose. Abscission and dehiscence in Arabidopsis. Plant Physiol., 126, 494-500.
5. Fernandez, D. E., Heck, G. R., Perry, S. E., Patterson, S. E., Bleecker, A. B. and Fang, S. C. (2000) The embryo MADS domain factor AGL15 acts postembryonically. Inhibition of perianth senescence and abscission via constitutive expression Plant Cell, 12, 183-198.
6. Jinn, T. L., Stone, J. M. and Walker, J. C. (2000) HAESA, an Arabidopsis leucine-rich repeat receptor kinase, controls floral organ abscission. Genes De.v, 14, 108-117.
7. Bleecker, A. B., Estelle, M. A., Somerville, C. and Kende, H. (1988) Insensitivity to ethylene conferred by a dominant mutation in Arabidopsis thaliana. Science, 241, 1086-1089.
8. Guzman, P. and Ecker, J. R. (1990) Exploiting the triple response of Arabidopsis to identify ethylene-related mutants. Plant Cell, 2, 513-523.
9. Patterson, S. E., Huelster, S. M. and Bleecker, A. (1994) Floral organ abscission in Arabidopsis thaliana. Plant Physiol, 105, 43.
10. Mandal, A., Sandgren, M., Holmstrom, K.-O., Gallois, P. and Palva, E. T. (1995) Identification of Arabidopsis thaliana sequences responsive to low temperature and abscisic acid by T-DNA tagging and in-vivo gene fusion. Plant Mol. Biol. Rep, 13, 243-254.
11. Kieber, J. J., Rotheberg, M., Roman, G., Feldmann, K. A. and Ecker, J. R. (1993) CTR1, a negative regulator of the ethylene response pathway in Arabidopsis, encodes a member of the Raf family of protein kinases. Cell, 72,427-441.
12. Chen, Q. G. and Bleecker, A. B. (1995) Analysis of ethylene signal-transduction kinetics associated with seedling-growth response and chitinase induction in wild-type and mutant Arabidopsis. Plant Physiol., 108, 597-607.
13. Roman, G., Lubarsky, B., Kieber, J. J., Rothenberg, M. and Ecker, J. R. (1995) Genetic analysis of ethylene signal transduction in Arabidopsis thaliana: Five novel mutant loci integrated into a stress response pathway. Genetics, 139, 1393-1409.
14. Grbic, V. and Bleecker, A. B. (1995) Ethylene regulates the timing of leaf senescence in Arabidopsis. Plant J., 8, 595-602.
15. Roberts, J. A., Whitelaw, C. A., Gonzalez, C. Z. H. and McManus, M. T. (2000) Cell separation processes in plants: Models, mechanisms and manipulation Ann. Bot. London, 86,223-235.

16. Mao, L., Begum, D., Chuang, H. W., Budiman, M. A., Szymkowiak, E. J., Irish, E. E. and Wing, R. A. (2000) JOINTLESS is a MADS-box gene controlling tomato flower abscission zone development. Nature, 406, 910-913.
17. The T-DNA of pMHA2 carries a marker gene (nptII) conferring Kanamycin (Km) resistance. The progeny of ida plants was always 100% Km resistant.
18. Genomic DNA flanking the Right border of the T-DNA was cloned using inverse PCR ((30), 1st primer set gus78 5′-CAC GGG TTG GGG TTT CT-3′ and gus 330 5′-TGC GGT CAC TCA TTA COG-3′, 2nd primer set gus 64L 5′-TTT CTA CAG GAC GGA CCA T-3′ and gus 342 5′-TTA CGO CAA AGT GTG GGT C-3′), while the other side was cloned by PCR with a T-DNA specific primer (5074+5′-ATT TGT CGT TTT ATC AAA ATG TAC-3′) and a genomic primer (ida49 5′ GGT GTT TCT ACT ATG CGT GTG 3′).
19. The fragments were inserted in the XbaI site of the Ti vector pGSC1704 (kindly provided by the Laboratory of Genetics, Flanders Interuniversity Institute for Biotechnology, Gent, Belgium) which has a Hygromycin resistant gene within the T-DNA. Ida plants were transformanted by the Agrobacterium tumefaciens-mediated floral dip method (31), and transformants were selected by germinating seeds on plates containing 10 mg/ml Hygromycin.
20. Agrobacterium tumefaciens will insert T-DNA at random positions in the plant genome. The expression level of a gene residing in T-DNAs will vary between independent transformants, due to position effects and/or integration of multi-copy or rearranged T-DNAs. We therefore assume that in the four transformants displaying the mutant ida phenotype although carrying the second construct, the expression level from the IDA gene was not sufficient to achieve complementation.
21. mRNA was isolated using Genoprep mRNA beads (Genovision, Norway) and treated with 1 U DNaseI (Invitrogen Cat. No 18068-015) per mg mRNA for 15 min. at room temperature, prior to first strand cDNA synthesis with AMV—Reverse Transcriptase (Promega). The IDA cDNA fragment of 421 bp was amplified by PCR using the primers pipp2U (5′ GAAGAAAAAAAACATTGACTCCA-3′) and pipp162 (5′-TGGCCGTAATGACCTTAAAC-3′). The ACTIN2-7 cDNA fragment of 294 bp was amplified using the primers 5′-GCTGGTTTTGCTGGTGATGATG-3′ and 5′-TAGAACTGGGTGCTCCTCAGGG-3′.
22. Chuang, C. F. and Meyerowitz, E. M. (2000) Specific and heritable genetic interference by double-stranded RNA in Arabidopsis thaliana Proc. Natl. Acad. Sci. U S A, 97, 49854990.
23. Wesley, S. V., Helliwell, C. A., Smith, N. A., Wang, M. B., Rouse, D. T., Liu, Q., Gooding, P. S., Singh, S. P., Abbott, D., Stoutjesdijk, P. A. et al. (2001) Construct design for efficient, effective and high-throughput gene silencing in plants. Plant J., 27, 581-590.
24. Nielsen, H., Engelbrecht, J., Brunak, S. and von Heijne, G. (1997) Identification of prokaryotic and eukaryotic signal peptides and prediction of their cleavage sites. Protein Engineering, 10, 1-6.
25. The coding region and the region encoding the first 24 aa encompassing the putative signal peptide, both with 96 bp 5′ UTR, were amplified with primers (idaGFPA 5′-TTATTCATTTCATTCATAAGACCCTTC-3′ plus idaGFPD 5′-ATGAGGAAGAGAGTTAACAAAAGAG-3′ and idaGFPA plus idaGFPE (5′-ACAAGAACTACTCGCCGC-3′, respectively) with additional Gateway att-sequences in the 5′ end and recombined into the vector pKEx4tr-smGFP (32) converted to a Gateway vector by the Gateway Vector Conversion System (Invitrogen), so that the IDA coding region or signal peptide would be expressed as fusion proteins with the Green Fluorescence protein (GFP) in the C-terminal end.
26. Rojo, E., Sharma, V. K., Kovaleva, V., Raikhel, N. V. and Fletcher, J. C. (2002) CLV3 is localized to the extracellular space, where it activates the Arabidopsis CLAVATA stem cell signaling pathway. Plant Cell, 14, 969-977.
27. Cock, J. M. and McCormick, S. (2001) A large family of genes that share homology with CLAVATA3. Plant Physiol., 126, 939-942.
28. Vanoosthuyse, V., Miege, C., Dumas, C. and Cock, J. M. (2001) Two large Arabidopsis thaliana gene families are homologous to the Brassica gene superfamily that encodes pollen coat proteins and the male component of the self-incompatibility response Plant Mol. Biol., 46, 17-34.
29. Shiu, S. H. and Bleecker, A. B. (2001) Plant receptor-like kinase gene family: diversity, function, and signaling, Sci. STKE, 2001, RE22.
30. Meza, T. J., Stangeland, B., Mercy, I. S., Sk\dot{a}rn, M., Nymoen, D. A., Berg, A., Butenko, M. A., H\dot{a}kelien,. A.-M., Haslek\dot{a}s, C., Meza-Zepeda, L. A. et al. (2002) Analyses of single-copy Arabidopsis T-DNA transformed lines show that the presence of vector backbone sequences, short inverted repeats and DNA methylation is not sufficient or necessary for the induction of transgene silencing Nucl. Acids Res., 30,4556-4566.
31. Clough, S. J. and Bent, A. F. (1998) Floral dip: a simplified method for Agrobacterium-mediated transformation of Arabidopsis thaliana Plant J, 16,735-743.
32. Baumbusch, L. O., Thorstensen, T., Krauss, V., Fischer, A., Naumann, K., Assalkhou, R., Schulz, I., Reuter, G. and Aalen, R. B. (2001) The Arabidopsis thaliana genome contains at least 29 active genes encoding SET domain proteins that can be assigned to four evolutionarily conserved classes. Nucl. Acids Res., 29, 4319-4333.
33. The manipulation and modification of tomato fruit ripening by expression of antisense RNA in transgenic plants.Picton-Steve {a}; Gray-Julie-E; Grierson-Don {a} Applied Biosystems Ltd., Kelvin Close, Birchwood Sci. Park North, Warrington, Cheshire WA3 7PB, UK Euphytica-. 1995; 85 (1-3)193-202. 1995

1. A method of decreasing organ abscission in a plant, comprising reducing the expression level of a gene in a plant, wherein the gene:
a) is the IDA gene or a mimetic thereof;
b) includes a coding sequence for a C terminal motif PpSa/gPSk/rk/rHN;
c) includes a coding sequence for a N terminal hydrophobic signal peptide;
d) includes a coding sequence for a C terminal motif v/iPpSa/gPSK/rk/rHN and a coding sequence for an N terminal hydrophobic signal peptide;
e) is selected from: AY087883, AtIDL1, gene At3g25655, tomato (LeIDL1, ac. no AI779570, lotus (LjIDL1, ac. NO. AW719486), soybean (GmIDL1 ac. NO. Bq630646), black locust (RpIDL1, ac. NO. BI642538), maize (ZmIDL1, ac. NO. BI430572), poplar (PtIDL1, ac. NO. BU889756), and wheat (TaIDL1, ac. no BM135459); or
f) is or comprises all or part of the nucleotide sequence presented as any one of SEQ ID No. 1, SEQ ID No. 2, SEQ ID No. 8, SEQ ID No. 9, SEQ ID No. 10 or SEQ ID No. 11 or a variant of any thereof or a homologue of any thereof.

2. A method according to claim 1 wherein said expression level is reduced by mutating said gene.

3. A method according to claim 1 wherein said expression level is reduced by mutating a regulatory region of said gene.

4. A method according to claim 1 wherein said expression level is reduced by mutating the promoter region of said gene.

5. A method according to claim 1 wherein said expression level is reduced by mutating the promoter region of said gene by use of T-DNA insertion techniques or by use of interfering moieties.

6. A nucleotide sequence wherein the nucleotide sequence is
a) a modified sequence;
b) all or part of a sequence selected from: AY087883, AtIDL1, geneAt3g25655, tomato (LeIDL1, ac. No. AI779570, lotus (LjIDL1, ac. NO. AW719486), soybean (GmIDL1, ac. NO. Bq630646), black locust (RpIDL1, ac. NO. BI642538), maize (ZmIDL1, ac. NO. BI430572), poplar (PtIDL1, ac. NO. BU889756), and wheat (TaIDL1, ac.no BM135459), a nucleotide sequence that includes a coding sequence for a C terminal motif PpSa/gPSk/rk/rHN, a nucleotide sequence that includes a coding sequence for an N terminal hydrophobic signal peptide, a nucleotide sequence that includes a coding sequence for a C terminal motif v/iPpSa/gPSK/rk/rHN and a coding sequence for an N terminal hydrophobic signal peptide; or
c) all or part of a nucleotide sequence according to b), further comprising one or more mutations in any of said sequence;
wherein when a plant comprises or is transformed with said nucleotide sequence said plant exhibits decreased organ abscission.

7. A nucleotide sequence according to claim 6 wherein said sequence is any one of SEQ ID No. 1, SEQ ID No. 2, SEQ ID No. 8, SEQ ID No. 9, SEQ ID No. 10 or SEQ ID No. 11 or a variant of any thereof or a homologue of any thereof or a mimetic of any thereof; and wherein said nucleotide sequence comprises one or more mutations in any of said sequence.

8. A nucleotide sequence according to claim 6 wherein said sequence is any one of SEQ ID No. 1, SEQ ID No. 2, SEQ ID No. 8, SEQ ID No. 9, SEQ ID No. 10 or SEQ ID No. 11; and wherein said nucleotide sequence comprises one or more mutations in any of said sequences.

9. A construct, vector, expression vector, transformation vector, host cell, organ, organism, or transformed plant comprising the invention according to claim 6.

10. A transformed plant according to claim 9 wherein said plant is a flowering plant or tree.

11. A method comprising transforming a plant with the invention according to claim 6.

12. A method of using the invention according to claim 6 in the preparation of a transformed plant to decrease organ abscission.

13. An isolated and/or purified nucleotide sequence, construct, vector, expression vector, transformation vector, or transformed cell comprising all or part of, or which can hybridise to or is complementary to, the nucleotide sequence presented as SEQ ID No. 1, SEQ ID No. 2, SEQ ID No. 8, SEQ ID No. 9, SEQ ID No. 10 or SEQ ID No. 11, or an isolated amino acid sequence comprising the sequence as shown in SEQ ID No. 3 or a sequence substantially homologous thereto, or a fragment thereof.

14. A promoter sequence comprising all or part of the nucleotide sequence of claim 13.

15. A method of using the promoter sequence of claim 14 to express an heterologous coding sequence.

16. The method according to claim 15 wherein said heterologous coding sequence is a mutated coding sequence corresponding to the coding sequence naturally associated with said promoter sequence.

17. A method if using the invention according to claim 6 in the preparation of a transformed plant to prevent or reduce plant cell degradation.


SEQUENCE LISTING (42 sequences)

SEQ ID NO: 1 - DNA, 2019 bp, artificial sequence
  IDA modified
ttctgaatcaaagggtttgtgataacaaaaacttggaatgatacaaagctgtggtcttttcaattttgttattgcatcttatttctatagcattattttgttttaaattatttagcgtatttacaaaatatagttttgatatctcgaagttcattagaaaaacttccaagtattttttttttagatcatacaatcataaaattaaataacagacttacctagtttaaatttactgtgtatcaatgattcaatgtatgatacggtcggttcttacctagttaaaatttgtttttttttagtatatgtaaagatctacctgtttccatcaaattagaagaattgtatgattttatgcgacatgtctaatcgtaggaggtatgttggaactttattttattttggcatttcataaaggaaatataaaaaattctatttaggtaaaaataataatccgtgactaagtccgatatagatgcagaaatcaggtggatatccaaatcaaatttggcataatttaaagaaaaatgtgaatcatggaaagccacttgtcaagttgtaaaattgtgtataactgagtcaaccctccaagctactttttgttctctctttaatttcttaaaataccttctcacgcaaaagatagagttggtatacattgtttttacgtgttttatgcatgcatttttcagtatattttatgtgtatattcgtttaactaaattattatttattccgtatcatgattaacctacgacatgttatcatttagtctaatccttttactatacatacgtgtattgtctaccactttcgtcattcgtccaaggatgatatctaattttctttctgcacgacatatatgatatattgtgtgcttaaagaaagctcatgagtcatgtctgatatttgagttcgaatttcgaaaactaatcttgctttactattcatacatcttgtgatatagttactgttgaaagttgaataatgcaaaagaaaacaaaagatagctcatggtgacgatgcctataaaaccattaattattcttcttcccgtcaacgtacttgtacatatatgatgaaatcttgaaaaaaacgaccatcccatcaaaccagattcccatttcctagcataaaatggacttctatagaaaaatacaaaggaaaataaaagtcgaaggcggtgactaaaacaacgagaaaaactcaaacctttgaaaactttagaattcttataaaataataaggaaaattcaaagtctcaatagcgtgggattcactccactcttaccattccccgacttccccctcatttccgccacacttatttccaatcaaatccacacacacacatgttataaatataacacatagtcacatacatacacattattcatttcattcataagacccttcattcatttactcaaagaagtggaagtgaaaaaattagaaaaagagaagaaaaaaaacattgactaccaaatggctccgtgtcgtacgatgatggttctgctctgttttgttctgtttctcgcggcgagtagttcttgtgtagcggctgcaagaattggagccaccatggagatgaagaagaatataaagagattaacgtttaaaaacagccatatttttggttacttacctaaaggcgttcccattcctccttctgctccttctaagagacacaactcttttgttaactctcttcctcattgatttcattttattttattcattgtattcttttaggtttggttttgtagattagatcaacatagggtattatactctttgtatatgcatacgatttatatttacttttgaatttgttcctttttatgtttatagacttaatatatgtttaaggtcattacggccatataaagatcaagaaatcgattaagctttgttagccttctactaaagaatattccatagacctcactcatcatacgctaatcaaacatatgtttttatgcaacttgtaagttatatcattcgtctttctcaaataggtca

SEQ ID NO: 2 - DNA, 536 bp, Arabidopsis sp.
attattcatttcattcataagacccttcattcatttactcaaagaagtggaagtgaaaaaattagaaaaagagaagaaaaaaaacattgactaccaaatggctccgtgtcgtacgatgatggttctgctctgttttgttctgtttctcgcggcgagtagttcttgtgtagcggctgcaagaattggagccaccatggagatgaagaagaatataaagagattaacgtttaaaaacagccatatttttggttacttacctaaaggcgttcccattcctccttctgctccttctaagagacacaactcttttgttaactctcttcctcattgatttcattttattttattcattgtattcttttaggtttggttttgtagattagatcaacatagggtattatactctttgtatatgcatacgatttatatttacttttgaatttgttcctttttatgtttatagacttaatatatgtttaaggtcattacggccatataaagatcaagaaatcgattaagctttgttagccttctac

SEQ ID NO: 3 - protein, 77 aa, Arabidopsis sp.
MAPCRTMMVLLCFVLFLAASSSCVAAARIGATMEMKKNIKRLTFKNSHIFGYLPKGVPIPPSAPSKRHNSFVNSLPH

SEQ ID NO: 4 - DNA, 286 bp, Arabidopsis sp.
atgtcgtctcgaaaccaaagatcaagaatcacctctagtttcttcgtctccttcttcactagaaccattcttcttcttctcattcttcttcttggtttctgcaatggagcaagaacaaacacgaacgtcttcaactcgaaaccacacaagaagcacaacgacgccgtttcttcttcaactaaacagttcttggggttcttgccacgtcacttccctgttccagcttctggtccctccagaaaacacaatgatattggtttacttagttggcatcgatcttctccgt

SEQ ID NO: 5 - DNA, 298 bp, Arabidopsis sp.
atgtcttctcgaagccaccgatcaagaaaatatcaactcacgagaacaattcctattcttgttcttcttcttgtccttttgagttgctgcaatggagcaagaaccacgaacgtcttcaacacaagctcgccacccaaacaaaaggacgtcgtttcgccacctcatgatcatgtccatcatcaagtacaagatcacaaaagtgtacagttcttggggtccctgccacgtcagtttccggttccaacttctggtccttccagaaaacataacgaaatcggtttaagtagtactaagacta

SEQ ID NO: 6 - DNA, 279 bp, Arabidopsis sp.
atgtatcctacacgtccacattattggagaagaagattatcaataaaccggccacaagcttttcttcttttgattctctgcctcttcttcattcaccactgcgacgcctcgagattctcgtcatcaagcgttttctacagaaaccctaattatgatcatagcaataacacggtgcggagaggccatttcttaggattcttgccaagacatttacctgttccagcttctgctccttcacgaaaacacaacgacattggtattcaagccttgttgtctcct

SEQ ID NO: 7 - DNA, 312 bp, Arabidopsis sp.
atgggcaataaacgcataaaagcaatgatgattctagtggtaatgatcatgatggtgttctcatggaggatttgtgaagccgatagcttaagaagatactcatcatcgtcaagaccacaaagatttttcaaggtgagaagacctaacccccgtaatcatcatcatcagaatcaaggtttcaatggtgatgattatcctccagaaagtttctcaggatttttgcccaagacattgccaattccacattctgctccttccaggaaacacaacgtctacggtttacagagtactaactcccatagatgtccatga

SEQ ID NO: 8 - DNA, 1482 bp, Arabidopsis sp.
ttctgaatcaaagggtttgtgataacaaaaacttggaatgatacaaagctgtggtcttttcaattttgttattgcatcttatttctatagcattattttgttttaaattatttagcgtatttacaaaatatagttttgatatctcgaagttcattagaaaaacttccaagtattttttttttagatcatacaatcataaaattaaataacagacttacctagtttaaatttactgtgtatcaatgattcaatgtatgatacggtcggttcttacctagttaaaatttgtttttttttagtatatgtaaagatctacctgtttccatcaaattagaagaattgtatgattttatgcgacatgtctaatcgtaggaggtatgttggaactttattttattttggcatttcataaaggaaatataaaaaattctatttaggtaaaaataataatccgtgactaagtccgatatagatgcagaaatcaggtggatatccaaatcaaatttggcataatttaaagaaaaatgtgaatcatggaaagccacttgtcaagttgtaaaattgtgtataactgagtcaaccctccaagctactttttgttctctctttaatttcttaaaataccttctcacgcaaaagatagagttggtatacattgtttttacgtgttttatgcatgcatttttcagtatattttatgtgtatattcgtttaactaaattattatttattccgtatcatgattaacctacgacatgttatcatttagtctaatccttttactatacatacgtgtattgtctaccactttcgtcattcgtccaaggatgatatctaattttctttctgcacgacatatatgatatattgtgtgcttaaagaaagctcatgagtcatgtctgatatttgagttcgaatttcgaaaactaatcttgctttactattcatacatcttgtgatatagttactgttgaaagttgaataatgcaaaagaaaacaaaagatagctcatggtgacgatgcctataaaaccattaattattcttcttcccgtcaacgtacttgtacatatatgatgaaatcttgaaaaaaacgaccatcccatcaaaccagattcccatttcctagcataaaatggacttctatagaaaaatacaaaggaaaataaaagtcgaaggcggtgactaaaacaacgagaaaaactcaaacctttgaaaactttagaattcttataaaataataaggaaaattcaaagtctcaatagcgtgggattcactccactcttaccattccccgacttccccctcatttccgccacacttatttccaatcaaatccacacacacacatgttataaatataacacatagtcacatacatacacattattcatttcattcataagacccttcattcatttactcaaagaagtggaagtgaaaaaattagaaaaagagaagaaaaaaaacattgactaccaa

SEQ ID NO: 9 - DNA, 1482 bp, Arabidopsis sp.; identical to SEQ ID NO: 8

SEQ ID NO: 10 - DNA, 1482 bp, Arabidopsis sp.; identical to SEQ ID NO: 8

SEQ ID NO: 11 - DNA, 2019 bp, Arabidopsis sp.; identical to SEQ ID NO: 1

SEQ ID NO: 12 - DNA, 234 bp, Arabidopsis sp.
atggctccgtgtcgtacgatgatggttctgctctgttttgttctgtttctcgcggcgagtagttcttgtgtagcggctgcaagaattggagccaccatggagatgaagaagaatataaagagattaacgtttaaaaacagccatatttttggttacttacctaaaggcgttcccattcctccttctgctccttctaagagacacaactcttttgttaactctcttcctcattga

SEQ ID NO: 13 - DNA, 21 bp, artificial sequence
  PCR primer
ttttcaattttgttattgcat

SEQ ID NO: 14 - DNA, 24 bp, artificial sequence
  PCR primer
atttggtagtcaatgttttttttc

SEQ ID NO: 15 - DNA, 17 bp, artificial sequence
  PCR primer
cacgggttggggtttct

SEQ ID NO: 16 - DNA, 18 bp, artificial sequence
  PCR primer
tgcggtcactcattacgg

SEQ ID NO: 17 - DNA, 19 bp, artificial sequence
  PCR primer
tttctacaggacggaccat

SEQ ID NO: 18 - DNA, 19 bp, artificial sequence
  PCR primer
ttacggcaaagtgtgggtc

SEQ ID NO: 19 - DNA, 24 bp, artificial sequence
  PCR primer
atttgtcgttttatcaaaatgtac

SEQ ID NO: 20 - DNA, 21 bp, artificial sequence
  PCR primer
ggtgtttctactatgcgtgtg

SEQ ID NO: 21 - DNA, 23 bp, artificial sequence
  PCR primer
gaagaaaaaaaacattgactcca

SEQ ID NO: 22 - DNA, 20 bp, artificial sequence
  PCR primer
tggccgtaatgaccttaaac

SEQ ID NO: 23 - DNA, 22 bp, artificial sequence
  PCR primer
gctggttttgctggtgatgatg

SEQ ID NO: 24 - DNA, 22 bp, artificial sequence
  PCR primer
tagaactgggtgctcctcaggg

SEQ ID NO: 25 - DNA, 27 bp, artificial sequence
  PCR primer
ttattcatttcattcataagacccttc

SEQ ID NO: 26 - DNA, 25 bp, artificial sequence
  PCR primer
atgaggaagagagttaacaaaagag

SEQ ID NO: 27 - DNA, 18 bp, artificial sequence
  PCR primer
acaagaactactcgccgc

SEQ ID NO: 28 - protein, 77 aa, Arabidopsis sp.
MAPCRTMMVLLCFVLFLAASSSCVAAARIGATMEMKKNIKRLTFKMSHIFGYLPKGBPIPPSAPSKRHNSFVNSLPH

SEQ ID NO: 29 - protein, 94 aa, Robinia pseudoacacia
MANYHSSKSLHLPCKTLSLVLLFVYLLLIGSCDAIRIGQTMKLNETREILKRKRNMQHGFPYKSMVFNFFPKGPVPPSGPSKRHNAVVDSTPNN

SEQ ID NO: 30 - protein, 95 aa, Lotus japonicus
MANSHSSKPLHLHRKTFSLILLLLYFLLIGSCTAIRTGATTKLKLNERREILQGKHNQQGFQYQSLVFNFFPKGRVPPSGPSKRHNAEVDSTPKN

SEQ ID NO: 31 - protein, 81 aa, Arabidopsis sp.
MQLPCRNFSLFLFFFFLLIGSCAAIRTEETMRMKMNERRDLRIPQGKHNTMMVFNFFPKGWVPPSGPSKRHNEVVDSTPQN

SEQ ID NO: 32 - protein, 101 aa, Lycopersicon esculentum
MAFSFSSSKTLYLSSKLTWLILVISLLFNYGHIVEASRFGRIMMVEENSRIFSSQHMKVYKKENAYKVDNLLFTMLPKGIPIPPSAPSKRHNAIDESTPQN

SEQ ID NO: 33 - protein, 94 aa, Glycine max; identical to SEQ ID NO: 29

SEQ ID NO: 34 - protein, 78 aa, Populus sp.
MMVACRRPLTLLLCLFFLFFIFFVGCSHGSRSTNVFNFKPKTQYKGHFLNFLPGHLPIPTSGTSRRHMGIGLQNWRSP

SEQ ID NO: 35 - protein, 89 aa, Zea mays
RRRGRRRPLLLAKKPPWRLVLAFLILMLVAPSCCQATARARGMQPFRGRPLLEPGSANRFLGFFPRGPVPPSGPSRQHNSIGAQDQSHP

SEQ ID NO: 36 - protein, 89 aa, Triticum sp.
MGRTSGRRTTLLPPQLRWYVLPLLLLILLAMASSSSCEASRGMQPFRGRPLERGAANHFFGFLPRGSVTPSGPSRKHNAVGLDSQLEKP

SEQ ID NO: 37 - protein, 95 aa, Arabidopsis thaliana
MSSRNQRSRITSSFFVSFFTRTILLLLILLLGFCNGARTNTNVFNSKPHKKHNDAVSSSTKQFLGFLPRHFPVPASGPSRKHNDIGLLSWHRSSP

SEQ ID NO: 38 - protein, 99 aa, Arabidopsis thaliana
MSSRSHRSRKYQLTRTIPILVLLLVLLSCCNGARTTNVFNTSSPPKQKDVVSPPHDHVHHQVQDHKSVQFLGSLPRQFPVPTSGPSRKHNEIGLSSTKT

SEQ ID NO: 39 - protein, 79 aa, Arabidopsis thaliana
MFMTLYIVFLLIFGSYNATARIGPIKLSETEIVQTRSRQEIIGGFTFKGRVFHSFSKRVLVPPSGPSMRHNSVVNNLKH

SEQ ID NO: 40 - protein, 77 aa, Arabidopsis sp.; identical to SEQ ID NO: 3

SEQ ID NO: 41 - protein, 93 aa, Arabidopsis thaliana
MYPTRPHYWRRRLSINRPQAFLLLILCLFFIHHCDASRFSSSSVFYRNPNYDHSNNTVRRGHFLGFLPRHLPVPASAPSRKHNDIGIQALLSP

SEQ ID NO: 42 - protein, 91 aa, Arabidopsis thaliana
MGNKRIKAMMILVVMIMMVFSWRICEADSLRRYSSSSRPQRFFKNQGFNGDDYPPESFSGFLPKTLPIPHSAPSRKHNVYGLQSTNSHRCP